(19) United States
(12) Patent Application Publication
(10) Pub. No.: US 2021/0275279 A1
(43) Pub. Date: Sep. 9, 2021

(54) ROBOTIC ASSISTED SCREW-ATTACHED PICK-UP DENTAL COPING SYSTEMS AND METHODS

(71) Applicants: Brandon Dale Kofford, Apex, NC (US); Charles Albert Rudisill, Apex, NC (US)

(72) Inventors: Brandon Dale Kofford, Apex, NC (US); Charles Albert Rudisill, Apex, NC (US)

(21) Appl. No.: 17/320,182

(22) Filed: May 13, 2021

Related U.S. Application Data
(63) Continuation-in-part of application No. 16/596,361, filed on Oct. 8, 2019.
(60) Provisional application No. 63/026,646, filed on May 18, 2020.
(60) Provisional application No. 62/818,082, filed on Mar. 13, 2019.
(60) Provisional application No. 62/774,402, filed on Dec. 3, 2018.
(60) Provisional application No. 62/742,942, filed on Oct. 9, 2018.

(51) Int. Cl. A61C 1/08; A61C 8/00; B25J 9/16

(52) U.S. Cl. CPC A61C 1/084; A61C 8/0001; A61B 34/30; B25J 9/1676; A61C 8/0089

(57) ABSTRACT

Systems and processes are disclosed for providing rapid and accurate dental prosthetic placement on implant abutments with robotic positioning for coping pick-up processing using a provisional fastener. In an embodiment, a dental arch with an open intaglio surface is bonded onto copings that are mounted onto threaded implant abutments with axially separable fasteners through a pick-up process using the same robotic system used to install the implant abutments into bone. The robotic system may apply movement to the prosthesis to aid its removal from abutments after the copings are bonded. The prosthesis with incorporated copings can subsequently be modified for attachment with definitive screws. One embodiment includes a prosthesis with integral robotic attachment features avoiding imaging of fiducial references for proper placement. An embodiment includes temporary arch indexing features between opposing arches to provide proper occlusion during pick-up processing.

### CROSS-REFERENCE TO RELATED APPLICATIONS

This disclosure is a continuation-in-part application of U.S. non-provisional patent application Ser. No. 16/596,361, filed on Oct. 8, 2019 which claims priority of U.S. provisional patent application No. 62/742,942, filed on Oct. 9, 2018, and U.S. provisional patent application No. 62/774,402, filed on Dec. 3, 2018, and U.S. provisional patent application No. 62/818,082, filed on Mar. 13, 2019. This disclosure claims priority to U.S. provisional application No. 63/026,646, filed on May 18, 2020. All of the above applications are incorporated herein by reference in their entirety.

### COPYRIGHT NOTICE

A portion of the disclosure of this patent application contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

Different systems have been introduced for attaching dental prostheses to dental implants to replace one or more natural teeth. In order to simplify future modification or replacement needs, it is desirable to have reversable attachment between the implants and prostheses using mechanical systems as opposed to directly bonding these components together. These systems require features to provide both proper alignment and retention for acceptable use by the patient. Intermediate components such as copings and separable abutment components having different lengths or orientations are often employed to provide proper registration between a dental prosthesis, one or more implants embedded in the patient's jawbone, and the soft-tissue and any remaining natural teeth. These intermediate elements may be mutually attached with screws or with ball-and-socket or other forms of snap-on mounts. In the case of a single-tooth crown attachment, the coping and abutment surfaces preferably include features to remove rotational symmetry in the mating of the abutment and coping surfaces. Rotational locking features may also be included in these single mount systems. When the prosthesis contains multiple copings for attachment to multiple abutments, this rotational fixation is not generally required. For example, 30 degree tapered mating surfaces for multiple interface locations are sufficient to provide complete registration.

It is common to attach a complete upper or lower denture to four or more implants. The multi-unit abutment system interfaces with mating surfaces of the final prosthetic. In some cases, these dentures may be designed or fabricated from scratch following the pulling of undesirable teeth and mounting of implants. In other cases, no teeth are pulled, but it is desirable to convert an existing removable denture to definitive mounting to new implants. In some cases, it is preferable to convert these existing dentures shortly after the implants are placed. This chair-side processing decreases patient discomfort by providing at least temporary dentures more quickly.

Dental impressions are often used to provide information on the location of implants, soft tissue and existing teeth for designing new prostheses or mounting copings in existing dentures for implant attachment conversion. Generally the copings are mounted to implant abutments and an impression is made to provide location information for the prosthesis through the copings. Impression material is introduced and cured around the copings to define their position. In the case of transfer copings, less rigid impression material is used to form a cavity for each coping; the coping remains attached to the abutment and the cured impression is removed. Subsequently, copings are inserted into the impression. In the case of pick-up copings, the coping is directly retained in the impression material after curing. That is, the copings are picked-up when the impression is removed from the mouth. The resulting alignment of pick-up copings is generally superior to transfer copings as a result of the direct transfer of coping location information. Transfer copings introduce an indirect secondary alignment reference since the insertion of the coping into the impression may not accurately duplicate the original position, particularly in insertion depth.

Pick-up copings can be used with either an open tray or a closed tray protocol. For snap-on systems, a closed impression tray pick-up technique may be done. The tray can be closed because there is no need to access the copings in order to disconnect them after the impression material sets. However, it is desirable to ensure that the assembly of snap-on copings can be removed without patient discomfort. The amount of force required to remove the tray or converted prosthesis depends upon the total number of implant abutment/coping sets and their position and mutual alignment. Some snap-on system provide features to provide different retention levels, but this complicates the installation process. Special tools have been introduced to help separate impression trays or prostheses with snap-on systems, but may still result in patient discomfort. Since snap-on systems are generally physically larger than dental screws, converting an existing denture may require large clearance cavities to be bored into the existing denture before it can be used as an impression tray in a pick-up coping process. These large holes may significantly reduce the mechanical stability of the existing denture. The mechanical precision required of snap-on system elements generally makes them more expensive than screw-attached systems.

The simplicity of screw-attached systems provides some benefits over snap-on systems beyond fabrication cost. The mounting pressure between the coping and abutment is readily controlled through the torque applied to the screw to tighten it. This axial tension control and the self-aligning characteristics of engaged screw threads provides more certainty in the engagement force and relative orientation of the components. Even if a screw breaks, techniques are known for removing the pieces without damage to surrounding components. Screws also have a benefit of independence for removal since each coping can be loosened individually. Tilting the prosthesis after screw removal to disengage one coping cannot cause reengagement of another coping.

A disadvantage with commercially available screw-attachment systems for pick-up copings is the requirement for using an open-tray impression in order to release the coping from the abutment after the impression material sets. By having an opening in the tray or existing denture, impression screws may be used. These impression screws extend through the tray beyond the impression material and can be unscrewed after the material sets. The impression screws use the same abutment threads as the semi-permanent screws used to attach the prosthesis later. As a result, the introduction of alignment uncertainty through a secondary reference may be avoided or minimized. The copings used with the impression screws are often relatively long and require modification for denture conversion. This customization may result in larger clearance holes in dentures and require additional process time by the dental practitioner. The length of the impression screws sticking through the tray can cause patient discomfort and a gag reflex. In the case of denture conversion, clearance for a set of impression screws at different angles may require additional material removal leading to a weakened denture. A schematic representation of the larger through-holes 136 required using conventional denture conversion processes for screw attachment are illustrated in FIGS. 53-54. The impression screws can also prevent the patient from applying bite pressure during the pick-up process to ensure proper registration of the modified denture and opposing teeth.

Prosthetic dentistry has rapidly moved into the realm of digital design and manufacturing. Typically, this requires that a digital model is created from a physical model or impression to allow digital design and manufacturing techniques to produce a physical prosthetic that can be delivered to a patient for restoration of dental and oral structures. When making a dental restoration with implants in this manner, an implant impression coping is often used in the mouth to reference the position of the implant geometry relative to surrounding structures such as gingiva, adjacent teeth, opposing teeth, etc. Once removed from the mouth, an implant analog is attached to the impression coping that was picked-up or transferred. With the lab implant analog attached to the impression coping in the elastomeric impression, dental stone is flowed into the impression and allowed to harden before separation from the impression with a resultant dental cast. A scan flag is attached to the lab analog on the dental stone model and is scanned in by laser or optical scanning technology. The scan flag is used for design software to reference and replicate the accurate positioning of the virtual implant relative to the adjacent teeth, gingiva and opposing tooth, as well as the timing of the implant and other pertinent implant geometries. Once the virtual implant is accurately brought into the design software, a prosthetic can be designed by following the workflow in the design software. A completed design is post-processed, and a CAD file is used in CAM software to direct either the 3-D printing or milling of the designed prosthesis. The manufactured prosthesis is verified on the physical model in a remote prosthetic manufacturing lab prior to delivery to the dentist or is verified directly on the dental patient if the prosthesis was manufactured in the local dental clinic. It is currently the standard of care to verify the fit, form, and function on a physical model.

Recently, an impression scan flag was introduced to the market for a few major implant systems and the most common multi-unit geometry. This scan flag allows for an impression to be digitized without the creation of a stone model. A digital model is created directly from the elastomeric impression, a prosthesis is designed digitally and processed with CAM software and both a 3-D printed model and 3-D printed or milled prosthesis is finished and tested on the 3-D printed model. The disadvantage of the 3-D printed model for full arch implant prosthetics is the positioning of the implant lab analog within the 3-D printed model introduces a degree of inaccuracy. Additionally, an impression only captures the implant or multi-unit abutment relative to the tissues and requires cumbersome steps to incorporate a provisional prosthesis or wax-up into the design software relative to the scan of the impression.

A properly converted denture provides a valuable source of information of the relative location of the implant and its abutment surfaces, copings and soft tissue and any remaining teeth of the patient. It can be used as a model for digital scanning to produce a duplicate denture if needed in the future or as a starting digital model for manipulation to improve aesthetics or other characteristics. However, if the mechanical integrity of the converted denture is compromised, the scanned information may not faithfully represent the relative geometry of these elements and result in a poor fit.

Other alignment systems have been proposed which use silicone or melting screw threads to allow closed tray transfer for definitive screw attachment, but the practicality of providing adequate alignment and seating forces with a screw diameter in the range of existing systems or overcoming the lack of disengagement independence required in melting all threads simultaneously without patient discomfort has not been documented. Details on removing any residual material in the abutment threads or in the prosthesis after the pick-up process have not been disclosed. Other hybrid systems that use a snap-on engagement for the pick-up coping during transfer and subsequent screw-attachment have also been proposed, but detailed information on the tradeoffs in precision and associated complexity or size required for equivalence performance to open-tray impression screw techniques have not been disclosed. A general need exists for systems that improve clinical efficiency, implant to prosthesis alignment accuracy, application to a wide range of coping designs and sources and patient comfort over existing systems.

In addition to the digital advances in prosthesis design and fabrication mentioned above, advances in dental implant surgery have also shortened the entire prosthesis delivery workflow from planning implant surgery, selecting appropriate implants, accurately drilling and incorporating implants into bone and providing temporary prostheses even on the same day. In particular, the use of a complete digital planning system with and robotic-assisted implant placement with flapless surgery has shortened the planning and surgical cycle times for orthodontic surgeons. At the same time, patient comfort is increased by smaller tissue disruption, reduced swelling and quicker healing cycles. The overall cycle time is improved by avoiding the development and production of drill guides and other patient specific fixtures.

These are significant benefits at the time of surgery; however, robot-assisted surgery has not responded to practically satisfy all of the needs to offer similar benefits to the prosthetic half of the treatment. The “crown-down” or “prosthetically-driven” surgical approach is widely accepted as the standard of care required for placing dental implants.

Systems have also been proposed for preparing prostheses by chairside milling using the same robotic system used for surgery. In some systems, necessary clearance holes for coping pick-up may be created in a prosthesis by replacing the implant drill bit with a boring bit, referencing the robotic actuator to the prosthesis and either assisting or autonomously creating clearance holes sufficiently large for allowing copings to be subsequently incorporated by the pick-up process. To avoid repeated test-fitting and provide sufficient bonding material between a prostheses and copings for successful pick-up, these clearance holes must be over-sized relative to the coping. When the prosthesis is presented to the patient, these oversized clearance holes may introduce inaccuracies in translational and angular placement. As noted previously for transfer copings, proper insertion depth may not be obtained. If these alignment inaccuracies are not minimized, the prosthesis may not be comfortable or aesthetically satisfying for the patient. Systems and methods for improving prosthesis positional accuracy or reducing workflow times or steps are desirable.

The present disclosure includes system and methods that address one or more of these issues in the prior art.

### BRIEF SUMMARY OF THE INVENTION

Some embodiments of the invention include a screw-in fastener system used to temporarily retain a coping on an abutment during bonding of the coping into a blind hole located in the tissue-side of a removable dental element such as a removable complete denture. The temporary fastener system disclosed has features to allow easy removal of the dental prosthesis from the fastener post after pick-up of the coping into the dental prosthesis is complete, without the need for special tools or access to the fastener system to unscrew it.

Some embodiments include a temporary fastener with a male-threaded bottom portion that is installed into the screw threads of an abutment, and an axially separable top portion, or cap, that retains the coping onto the abutment. The separable portion allows removal of the coping picked-up in the dental prosthesis without requiring mechanical access to the fastener. The inventive concepts disclosed facilitate easier installation and removal on multiple abutments that may be oriented at different compound angles when compared to the conventional open-tray processes and long impression screws. The temporary fastener may engage the same screw threads of the abutment that are used to definitively attach the prosthesis. In this manner, the coping is held against the abutment for the pick-up process with a force oriented identically to that of the final screw mounting. The retention of the cap on the screw post can be designed to provide a desired force holding the coping to the abutment through the applied torque on the temporary fastener. The cap can be attached to the post through a combination of known mechanical attachment means including but not limited to interference fits, adhesives, snap fits or other elastic and inelastically deforming retention elements. The cap may also be formed as an integral part of the screw post that has portions that fracture as a result of an applied axial force. In addition to applied axial forces, the cap may separate from the post as a result of retention changes resulting from chemical or thermal processes and or the application of electromagnetic energy during or subsequent to the pick-up process. In addition to steady axial forces, rocking or vibrational motion may also be applied to aid movement resulting in separation of components in the pick-up process.

Other embodiments include a threaded post that has a temporary retention feature that engages the coping without a separable cap. These embodiments engage the abutment threads in mounting the coping, but release the coping after bonding to the prosthesis.

The disclosed systems and methods can be applied to single tooth prostheses with copings and abutments with orientational features or for multiple tooth prostheses using copings and abutments with symmetrical mating surfaces such as 30 degree tapers.

In some embodiments, the temporary fastener is driven into the abutment threads by a tool that engages the post. In other embodiments, the temporary fastener is driven into the abutment threads by a tool that engages the cap. The drive torque can be designed to be sufficient for holding the coping to the abutment accurately, but less than the torque that would result in movement of the cap relative to the post.

In some embodiments, the threaded end of the post portion of the temporary fastener has a deflecting feature that allows the post to engage or disengage the abutment threads through axial motion instead of a rotary screw motion.

One embodiment describes a system for aligning a dental implant abutment, coping and separable dental element for definitive screw-attachment comprising:
a temporary alignment fastener comprising:
a post having an axis, a first post end and second post end, wherein the first post end is threaded for screw attachment to the implant abutment;
a cap, wherein the cap is attached to the second post end;
   and wherein the temporary alignment screw is configured to hold the coping against the implant abutment when the first post end is screwed into the implant abutment and wherein the cap is separable from the post through a release force directed away from the first post end.

One embodiment describes a system for aligning a separable dental element for installation to a threaded implant abutment with a definitive screw having a head and threaded shaft portion comprising:

a coping having a distal end shaped to engage with the implant abutment and a proximal end with an aperture sized to allow the shaft portion of the definitive screw to pass through;
a temporary screw having a distal end portion adapted to engage the threads of the implant abutment and a proximal end portion having temporary engagement means for attachment adjacent to the proximal end of the coping wherein the temporary screw holds the distal end of the coping in alignment against the implant abutment when the distal end portion of the temporary screw engages the implant abutment threads and wherein the temporary engagement means releases the coping without disengaging the post portion of the temporary screw upon the application of a predetermined axial force in the proximal direction.

In some embodiments, an existing removable denture is converted to a screw attached denture by milling pockets to allow copings to be bonded in a pick-up process. In this process, the denture acts in an equivalent manner to a closed impression tray. Proper registration can be confirmed by having the patient bite down on the denture to engage the opposing denture or teeth during the pick-up process. After coping pick-up, clearance for the definitive screws can be drilled using the coping as a guide. The converted denture can be used with scan flags to provide a 3-dimensional digital capture. Thus a digital model can be used to make a duplicate prosthesis or as the starting point for a new custom prosthesis.

One embodiment describes an alignment system for converting an existing denture for screw attachment to threaded implant abutments designed to perform the process of:
mounting pick-up copings to the implant abutments with temporary fasteners,
adhering the copings to cavities formed in the existing denture,
pulling the denture with secured pick-up copings away from the implant abutments,
unscrewing the threaded portions of the temporary fasteners from the implant abutments,
forming definitive screw clearance holes in the denture,
mounting the denture to the implant abutments with definitive screws that engage the same implant abutment threads as the temporary fasteners, wherein the unscrewing the threaded portions of the temporary fasteners and the forming definitive screw clearance holes in the denture may occur in either order.

In some cases there is no pre-existing denture to be modified for semi-permanent attachment. In some cases, a custom denture for the patient may have been designed and fabricated in advance of a scheduled implant surgery date. The custom denture may include oversized clearance bores for accommodating the copings and provisional fasteners in a planned pick-up process. Due to real-time surgical variations, it may be desirable to delay providing clearance bores until surgery is completed. The new custom denture may be processed for coping pick-up in an analogous manner to the existing denture conversion. The final modification of the new denture to remove excess material that would interfere with the coping pick-up hardware can be performed chairside manually by the prosthodontist or through robotic methods.

In the case of an emergency, implant surgery may be desirable without the delay for fabricating a custom prosthesis. In other cases, overall natural tooth replacement timing or expense may favor the use of the best fit from a selection of mass-produced dentures. The disclosed provisional fasteners and methods may be used to create clearance bores as summarized above. These clearance bores provide a dry-fit opportunity prior to pick-up, but there are tradeoffs in ensuring the most accurate positioning accuracy with manual presentation for pick-up processing, even when digitally controlled boring is used.

An embodiment is presented in which the denture with clearance holes bored is temporarily-attached to a mechanical bite fork with robotic arm interface. Fiducial elements in the bite fork allow scanning to reference the prepared prosthesis to the robotic system. The robotic system is then used to position and hold the prepared prosthesis to the implant abutment systems until the bonding material sets for the pick-up process.

An embodiment provides a method of utilizing a robotic arm or actuator with haptic technology to position a dental arch in the patient's mouth for precision pick-up of Ti bases or other temporary cylinders or copings according to the virtual positioning of the arch in the implant planning software. Custom or prefabricated dental arches may be used. Cost savings are provided by using mass-produced prefabricated stock dental arches. In embodiments in which the robot is used to guide the placement into the patient's mouth and maintain proper positioning for the bonding material to set, the robot can also be used to limit motion of the prosthesis during separation from the patient. With a complete digital model of the implants and prosthesis components and the patient's mouth, the robotic system can determine the optimum separation force vector or sequence of vectors to release the prosthesis. By limiting the motion of the prosthesis assembly, the robot can assist the dental practitioner in helping prevent contact with opposing teeth upon separation. The robot can also apply repetitive motion in programmable amplitudes and directions to aid separation of the structures used to hold the coping onto the abutment for pick-up. In addition to vibration or other forms of movement, the robotic actuator can be adapted to apply other forms of energy such as thermal or electromagnetic directly to the prosthesis to assist in coping separation from the implant abutment.

An embodiment is presented in which the prosthesis does not need to have material removed to create clearances for the copings and provisional fasteners. The prosthesis includes an intaglio side that resembles the trough of a closed impression tray. Bonding material filling the trough provides sufficient mechanical stability for temporary or semi-permanent installation. An embodiment includes robotic control of prosthesis positioning for bonding the prosthesis to the pick-up copings. A variant of this embodiment delays pick-up and prosthesis modification for definitive screw attachment; that is, the prosthesis is bonded in place with pick-up copings, but the axial separation of the provisional fasteners is avoided until a future dental appointment. In a complete digital planning and implementation process, the appropriate volume of bonding material can be calculated and dispensed by the complete digital design and robotic system. The additive material process reduces overall cycle times by avoiding multiple boring and test fitting cycles.

An embodiment is presented that includes a prosthesis that is supplied with an integral robotic arm interface. Fiducial markers or imaging to orient the prosthesis to a bite fork is not necessary for accurate placement. The prosthesis may be released from the arm in situ after bonding or it may be released after pick-up of the copings, as desired. The use of mass-produced prostheses of different sizes supplied with attached robotic interfaces and digital models reduces the overall design and prosthesis installation workflow. In an embodiment, an opposing prosthesis includes temporarily attachment means to ensure proper occlusion after bonding of pick-up copings. In an embodiment, the opposing prostheses are bonded to upper and lower copings simultaneously. In an embodiment, a robot arm is attached to and used to properly position one prosthesis, while the temporary attachment between the two prostheses positions the second prosthesis in proper occlusion with the first prosthesis.

Although axially-separable provisional screw-attached prosthesis are used to illustrate inventive concepts, some embodiments disclosed herein are not dependent upon the use of axially-separable provisional screws. For example, the pick-up processes and systems employing robotic positioning may also be advantageously employed with alternate coping attachment systems. Unless explicitly excluded, adaptation of embodiments illustrated with screw-attachment to non-screw attachment implementations is considered to be part of the scope of this application.

For the purposes of this disclosure, a separable dental element is defined to be anything that incorporates one or more dental copings that can be mounted and removed from one or more implant abutments. Different coping designs are known in the dental industry, and the systems and methods disclosed here can be adapted to work with many commercially-available types of copings including pick-up copings, temporary cylinders, inserts and impression copings. Implant abutments are known in the dental industry having compatible interfaces to these copings. For the purposes of this disclosure, an implant abutment system is defined to be any combination of hardware that comprises a dental implant or an intermediate component that is attached to a dental implant for positioning or attaching a prosthesis. During a pick-up process, a portion of an implant abutment system such as a coping or a portion of a separable fastener may become incorporated into a prosthesis to allow separable fastening in the same position. Since the mechanical interface is the same, for the purposes of this disclosure implant abutment is considered a generic term that includes abutment analogs. Description of alignment systems and process methods with copings and implant abutments that are installed in a patient's jaw should be considered to also describe equivalent inventive concepts that may be used with copings and implant abutment analogs in a dental lab. The inventive concepts disclosed herein can be used with different types of separable dental elements. The separable dental element can be any form of impression used in a dental lab to assist in creating and testing dental prostheses. A separable dental element can also be a dental prosthesis fabricated in the dental lab using a physical model made from the impression, a dental prosthesis newly fabricated, or an existing prosthesis being converted for screw attachment. A dental prosthesis is defined to include a single-tooth appliance such as a crown, or any multiple-tooth bridge, arch or denture. These prostheses may incorporate copings to provide a separable interface to provide orientation with an appropriate abutment attached to a patient's jaw or gingiva. The abutments for use with many of the inventive concepts disclosed herein include screw threads to mount the prosthesis with copings onto the abutments. While the concepts describe female threads in the abutment mating with male threads on a mounting screw, this is for convenience in disclosure. The inventive concepts could be applied with systems having male threads in the abutment engaging a screw with female threads for mounting the prosthesis. These are considered to be straightforward variations of the inventive concepts. Some inventive concepts may be applied to abutments not having screw threads.

For the purposes of this disclosure, the abutments may be integral to the implants embedded into the patients jaw or gingiva, or they may be separate units that are attached to the implants. The inventive concepts disclosed apply to both configurations.

For the purposes of this disclosure, an existing denture should be interpreted broadly to include any prosthesis that has been created prior to the use of the innovative systems and methods disclosed. An existing denture may be a loose denture that was worn by the patient prior to the installation of implants, or it may be a new denture that is in the process of first fitting in the patient's mouth.

The systems and methods disclosed herein can be used with prostheses for attachment to both the upper and lower jaw. As a result, portions of the system that are oriented downward for the lower jaw will be oriented upward for the upper jaw and vice versa. For convenience, a disclosure of an embodiment of inventive concepts that is limited to a single jaw orientation, is considered to disclose an embodiment for the opposite jaw orientation. When referring to the perspective of a clinician, proximal portions are nearer to the clinician than distal portions. While a term such as top is the opposite of the term bottom, and proximal is the opposite of distal, their actual relative orientation will be determined by the context of their use. The term tissue-side is used interchangeably with intaglio to indicate the side of a prosthesis that is opposite the occlusal or cameo surface.

The inventive systems disclosed are beneficially applicable to screw-attached prostheses. Key benefits of screw-attachment are variable tightening torques and reversibility. The terms permanent, semi-permanent, definitive and final when referring to screw-attachment are used interchangeably in this disclosure. A conventional screw that is definitively attached can still be removed by accessing the screw and rotating it in the opposite direction that was used for attachment. For the purposes of screw-attached prostheses for this disclosure, the attachment is semi-permanent, permanent or definitive in the sense that frequent attachment and removal is not anticipated for normal use. In contrast, the temporary screw attachment is applied for a planned process duration or other anticipated interval. Removal of a semi-permanent or definitive screw is generally motivated by a problem or an opportunity for an improvement. Access to the screw to apply a tool for removal may require removal of material covering the screw for aesthetic reasons.

Some of the inventive concepts disclosed include accessories and process flows of robotics systems. For brevity, a dental implant robotic system with a positional reference arm mechanically attached to the patient is used in the descriptions. The concepts can be used with other robotic systems and reference techniques known in the industry. Since understanding the concepts are generally not dependent upon specific details of the software and hardware of robotic systems, the robotic systems and functionality are not described in detail. Although completely integrated workflows containing a digital analog of all of the actual prosthetic elements, the patient, relative positions and movements at different times in the process are known, the inventive concepts can generally be applied to selected portions of the overall digital planning and physical processing environments. These digital planning and surgical workflow systems require knowledge of the physical dimensions, tolerances and relative positioning of elements of the patient, prosthetic components and tools. Some of these digital models and relationships are known through specifications used to manufacture them, while others are measured through various scanning and imaging techniques. The inventive concepts are generally not dependent upon how this mechanical information is obtained. A description of a process that includes imaging to determine the spatial relationship of two elements will be considered to also disclose a process in which digital models of individual components and a deterministic physical interface between them is used to determine spatial relationships without scanning.

For the purposes of this disclosure, the term “robotic assistance” should be interpreted to broadly cover different machine-human interactions in which a machine is programmed to provide information to a human performing a process or in which the machine performs a process autonomously when instructed by the human or hybrid processes, for example, in which the machine limits the range of motion of a device manipulated by the human. The communication between the machine and the human may include traditional keyboard, touchpad or joystick input, haptic feedback, sound input or output, optical sensing or feedback, or machine input/output or human sensory interaction. One form of robotic assistance in this disclosure is “spatial awareness”, in which a machine uses knowledge about the location and orientation of different physical entities relative to one or more portions of a machine to determine the relative location of different physical entities to one another. For example, a robot can be spatially aware of the relative position and orientation of an implant abutment and a prosthesis by processing known spatial relationship between the implant abutment and a first portion of the robot, the spatial relationship between the prosthesis and a second portion of the robot, and the spatial relationship between the first and second portions of the robot.

An embodiment is presented of a robotic assisted prosthesis delivery method comprising:
affixing a coping to an implant abutment with a means for provisional fastening,
mounting a prosthesis onto a robot,
applying a bonding agent to at least one of the coping or the prosthesis,
positioning the prosthesis on the coping in a predetermined position,
maintaining the relative position of the prosthesis and coping with robotic assistance for a predetermined minimum time for bonding the coping to the prosthesis.

An embodiment is presented of a robotic-assisted system for presenting a prosthesis to an implant abutment comprising:

a robotic system,
a delivery jig attached in a known spatial relationship to the robot,
a prosthesis, wherein the prosthesis is attached to the delivery jig in a known position, and
wherein the robotic system is spatially aware of the difference between the current position of the prosthesis to a predetermined mounting position of the prosthesis on the implant abutment system.

Other terms in the specification and claims of this application should be interpreted using generally accepted, common meanings qualified by any contextual language where they are used. The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically. The terms “about” and “essentially” mean±10 percent. Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation. The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive. When a process is described as including or comprising certain steps, steps that are not necessarily dependent upon the prior completion of other steps may be completed in any order.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting. Other objects, features, embodiments and/or advantages of the invention will be apparent from the following specification taken in conjunction with the following drawings.

### BRIEF DESCRIPTION OF THE DRAWINGS

FIG. 1 is a top exploded isometric view of a first embodiment of a system for aligning a dental implant abutment, coping and prosthesis for definitive screw-attachment.

FIG. 2 is a side plan view of a first embodiment of a system for aligning a dental implant abutment, coping and prothesis for definitive screw-attachment.

FIG. 3 is a top plan view of a first embodiment of a system for aligning a dental implant abutment, coping and prosthesis for definitive screw-attachment.

FIG. 4 is a side cross-sectional view of the system for aligning a dental implant abutment, coping and prosthesis for definitive screw-attachment along the line indicated in FIG. 3.

FIG. 5 is a side cross-sectional view of the embodiment of FIG. 4 attached to the jaw and prosthesis prior to the pick-up process.

FIG. 6 is a side cross-sectional view of the embodiment of FIG. 5 after the pick-up process.

FIG. 7 is a side cross-sectional view of the embodiment of FIG. 6 showing a drill bit creating a pilot hole for a definitive screw.

FIG. 8 is a side cross-sectional view of the embodiment of FIG. 7 showing a stepped drill bit creating a clearance for the head and shaft of a definitive screw.

FIG. 9 is side cross-sectional view of the embodiment of FIG. 8 showing the definitive screw holding the prosthesis to the implant.

FIG. 10 is an exploded top isometric view of a second embodiment of a system for aligning a dental implant abutment, coping and prothesis for definitive screw-attachment illustrating a single abutment and a temporary screw with breakaway tool.

FIG. 11 is an exploded side isometric view of the second embodiment of FIG. 10 prior to installation to the implant abutment.

FIG. 12 is an exploded side isometric view of the embodiment of FIG. 10 with the breakaway tool pushed onto the post and the coping placed on the implant abutment.

FIG. 13 is an exploded side isometric view of the embodiment of FIG. 10 schematically showing the tool being rotated to screw the post into the implant abutment.

FIG. 14 is an exploded side isometric view of the embodiment of FIG. 10 schematically showing the tool breaking away from the cap after the coping is secured on the abutment.

FIG. 15 is an exploded side isometric view of the embodiment of FIG. 10 schematically showing the prosthesis being marked for positioning a clearance hole.

FIG. 16 is a side isometric view of the marked prosthesis from FIG. 15.

FIG. 17 is a side isometric view of the marked prosthesis from FIG. 16 with blind clearance hole and boring tool.

FIG. 18 is an exploded side isometric view of the prepared embodiment of FIG. 17 as adhesive is schematically applied to the coping fixed to the abutment with the temporary screw.

FIG. 19 is a side view of the prosthesis in position for curing of the adhesive applied in

FIG. 18.

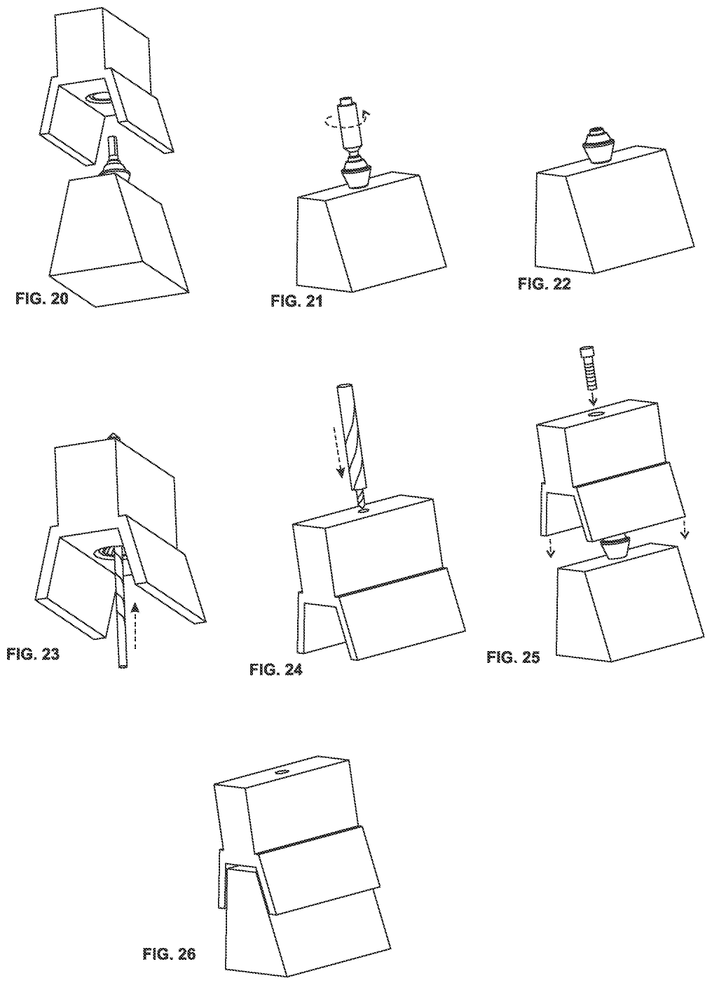

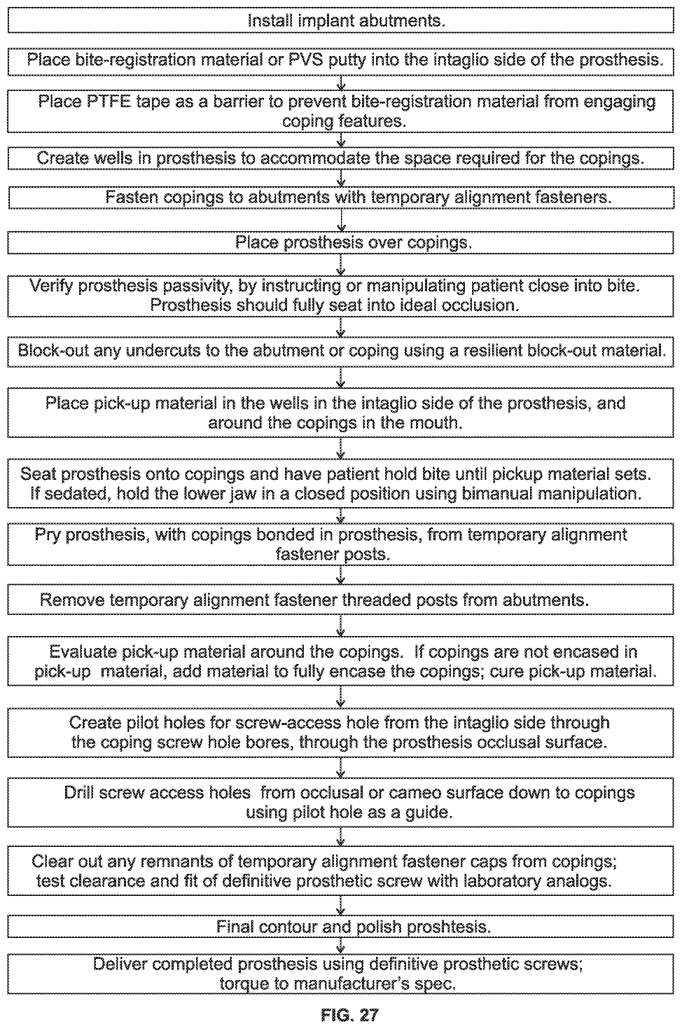

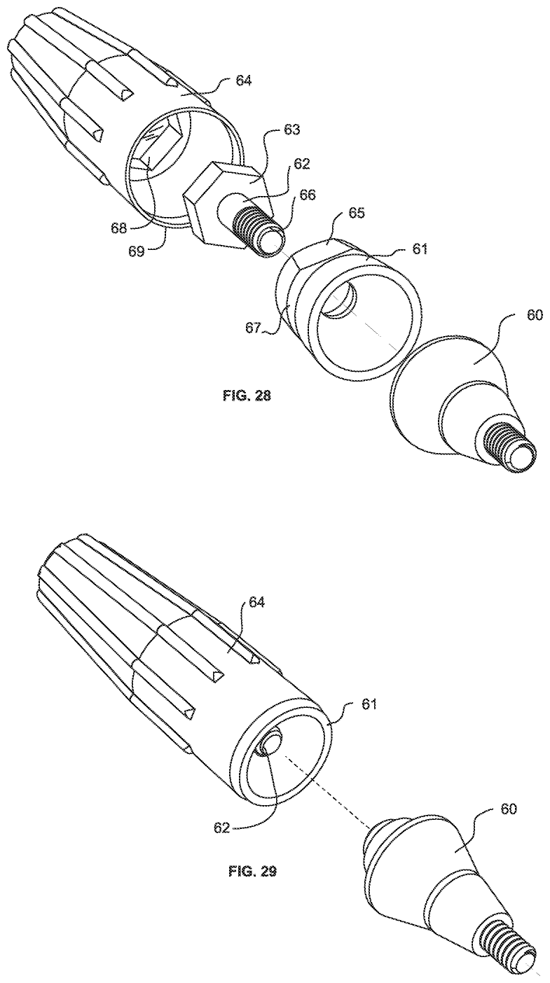

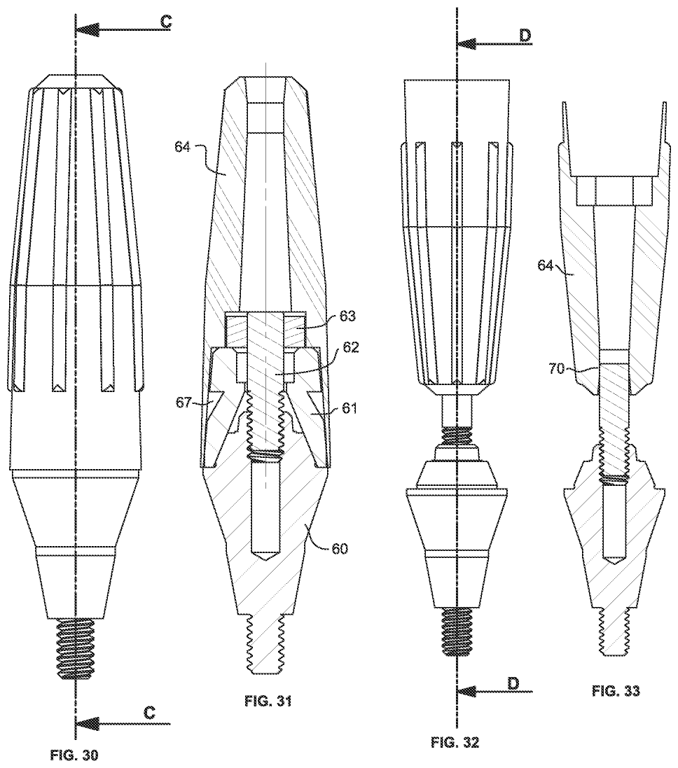

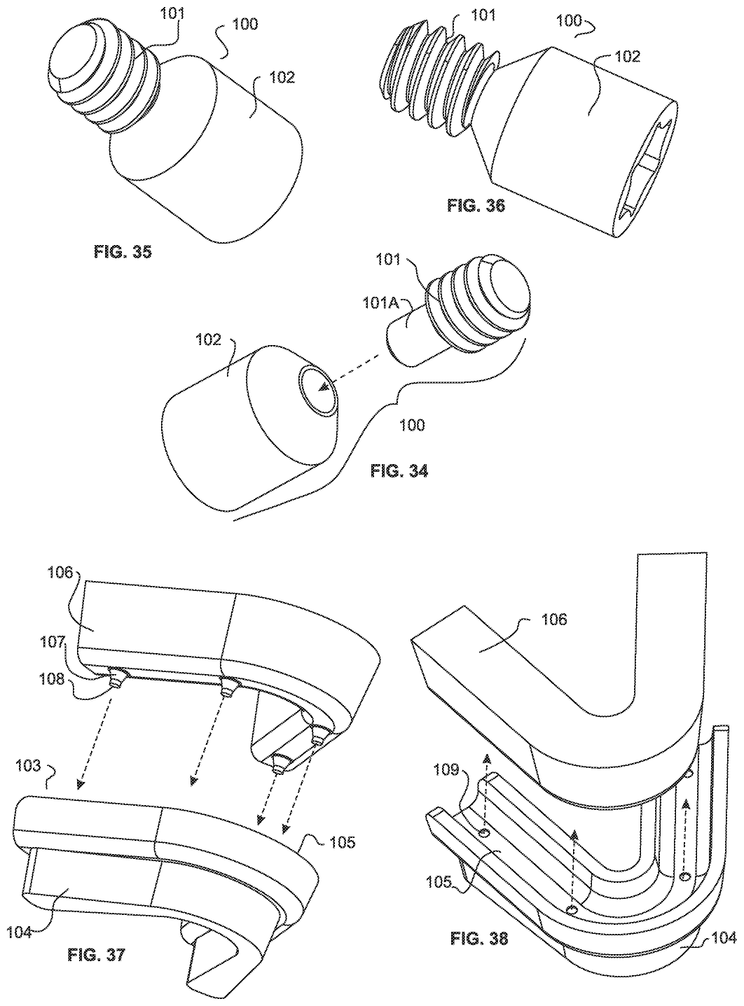

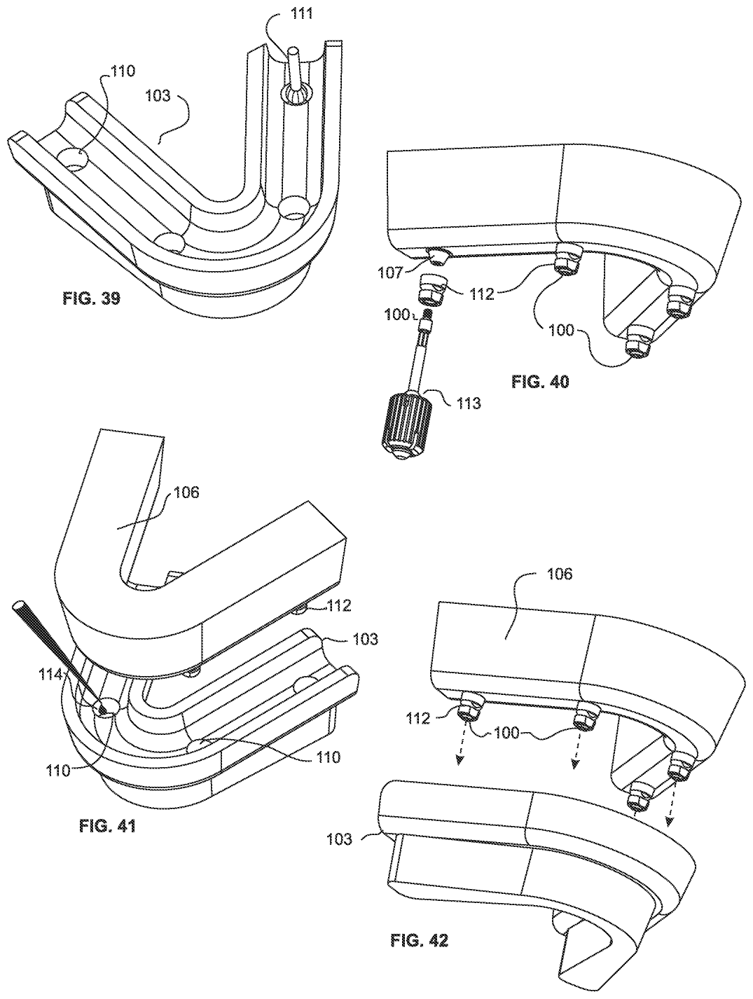

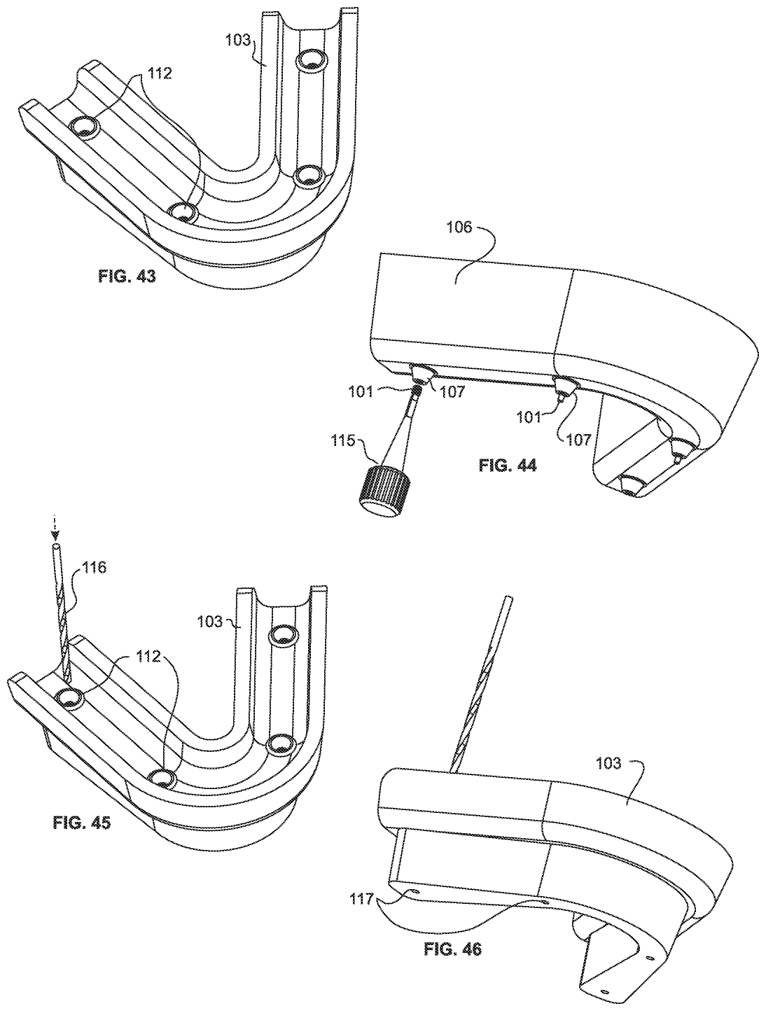

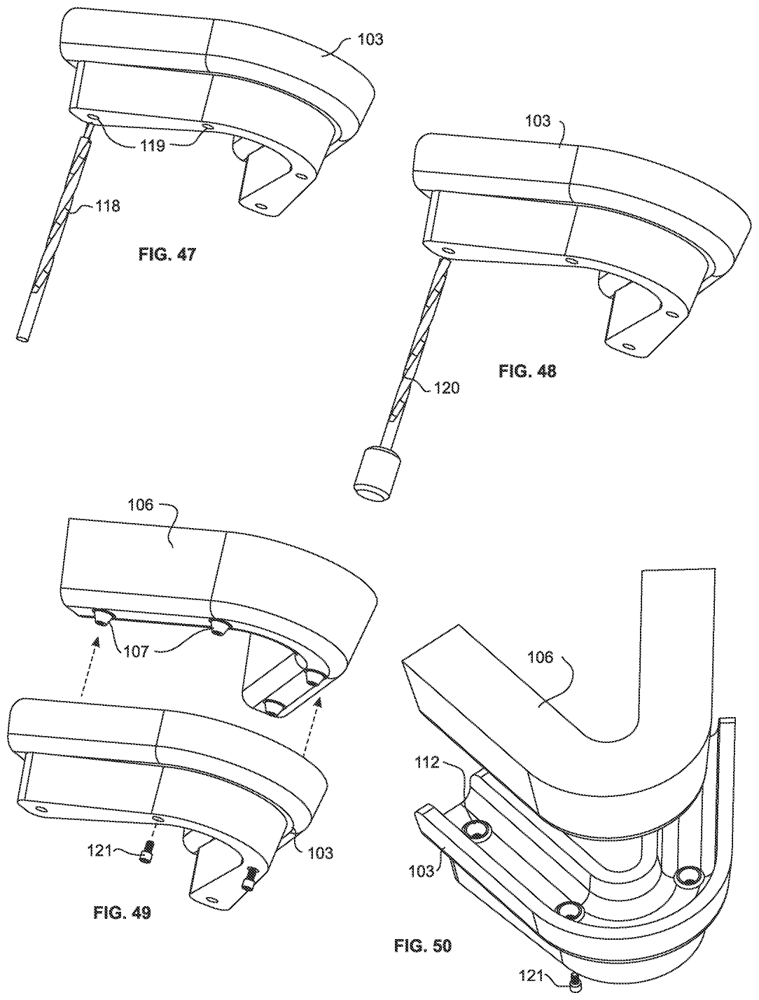

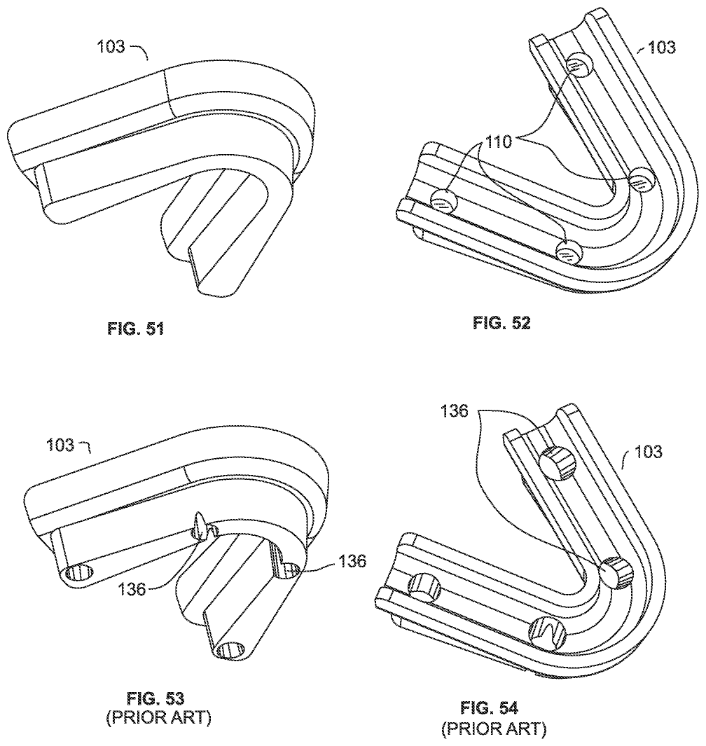

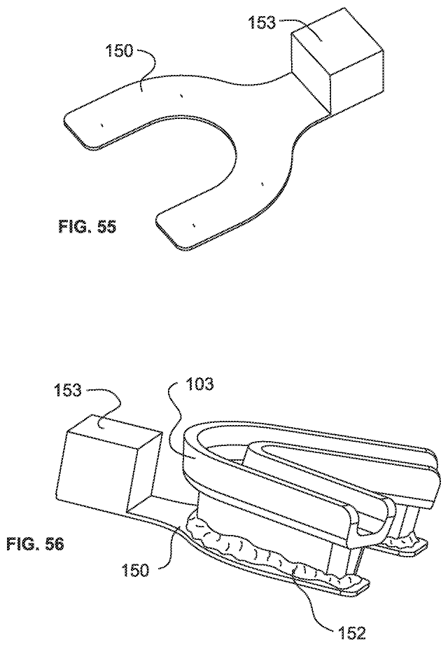

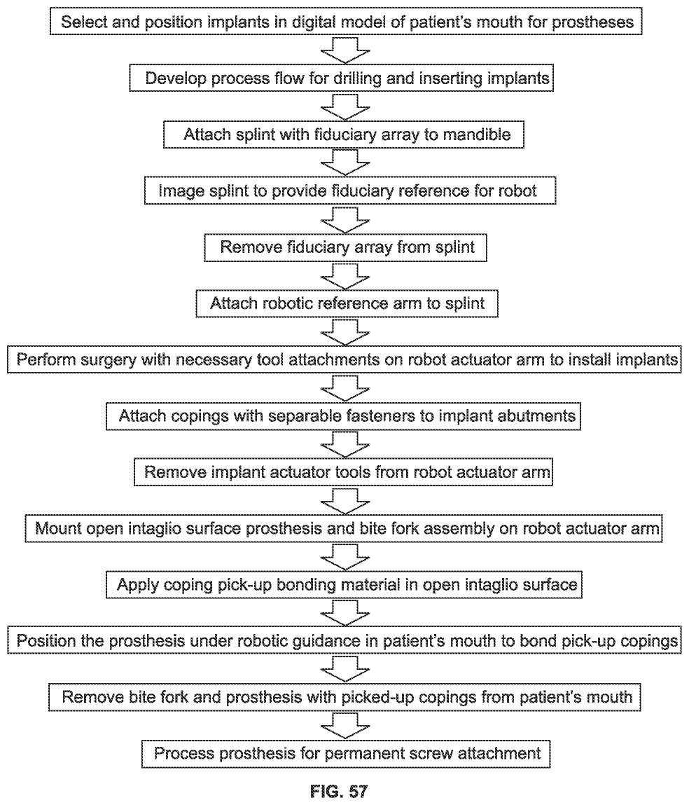

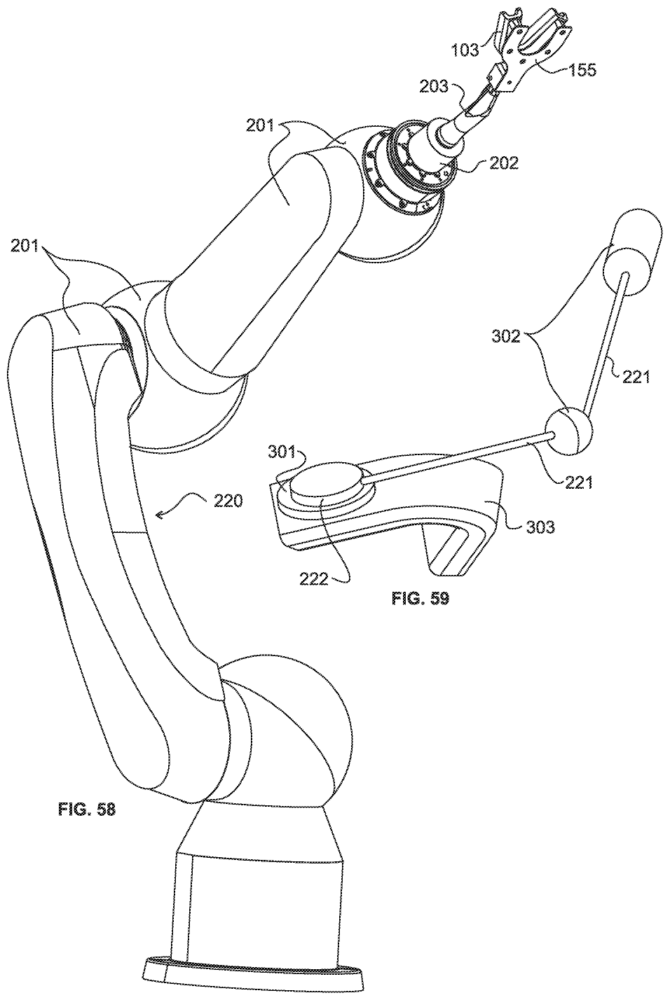

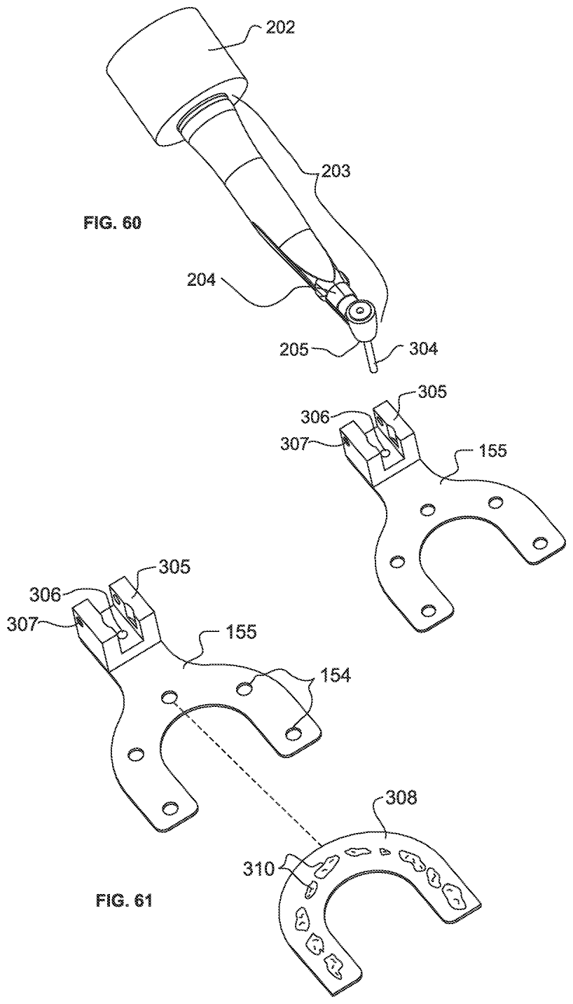

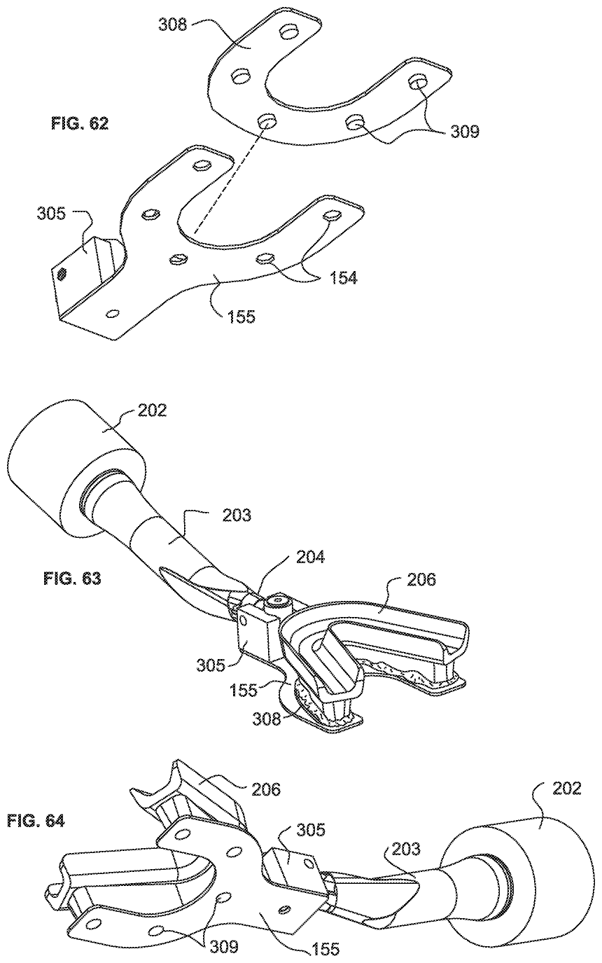

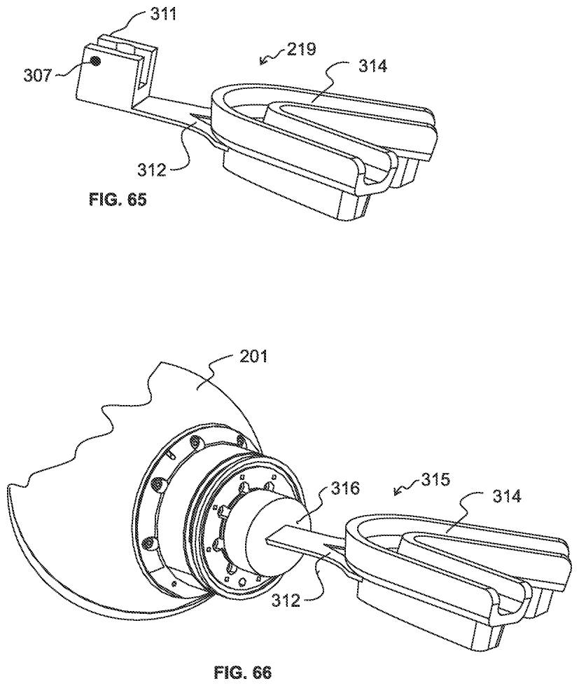

FIG. 20 is a bottom side isometric exploded view of the coping incorporated in FIG. 19 being picked up as the prosthesis is removed from the implant abutment.

FIG. 21 is a top side isometric view schematically showing the post of the temporary screw being unscrewed from the implant abutment.

FIG. 22 is a top side isometric view of the implant abutment prepared for attachment of a prosthesis with a definitive screw.

FIG. 23 is a bottom isometric view of the prosthesis with picked-up coping from FIG. 20 being drilled from the bottom to provide clearance for the threaded shaft of a definitive screw following coping pick-up.

FIG. 24 is a top isometric view of the prosthesis with picked-up coping from FIG. 20 with a drill applied from the top to provide clearance for the definitive screw head.

FIG. 25 is top exploded isometric view of the prepared prosthesis from FIG. 24 with the definitive screw.

FIG. 26 is a top isometric view of the prepared prosthesis from FIG. 24 after attachment to the implant abutment with the definitive screw.

FIG. 27 is a schematic description of a process for aligning dental implant abutments, copings and a prosthesis for definitive screw-attachment.

FIG. 28 is an exploded bottom isometric view of a third embodiment of a system for aligning a dental implant abutment, coping and prosthesis for definitive screw-attachment with tool oriented for assembly.

FIG. 29 is an assembled bottom isometric view of the embodiment of FIG. 28 with temporary screw and coping inserted into tool.

FIG. 30 is a side view of the system of FIG. 29 after assembly with the implant abutment.

FIG. 31 is a cross-sectional view of the assembly of FIG. 30 along C-C.

FIG. 32 is a side view of the assembly of FIG. 28 after the pick-up process showing the removal end of the tool engaging the retained temporary screw post.

FIG. 33 is a cross-sectional of the assembly of FIG. 32 along D-D.

FIG. 34 is an exploded isometric view of another embodiment of a temporary screw of a system for aligning a dental implant abutment, coping and prosthesis for definitive screw-attachment.

FIG. 35 is an isometric view of the temporary screw of FIG. 34 from the screw thread end.

FIG. 36 is an isometric view of the temporary screw of FIG. 34 from the cap end.

FIG. 37 is a bottom isometric view of a jaw with multiple abutments in preparation of marking a prosthesis as part of the process for aligning a dental implant abutment, coping and prosthesis for definitive screw-attachment.

FIG. 38 is a top isometric view of the prosthesis of FIG. 37 showing abutment location markings.

FIG. 39 is a top isometric view of the prosthesis with clearances milled to receive copings for pick-up.

FIG. 40 is a bottom isometric view showing installed copings and the assembly of a coping to an implant abutment with a temporary screw and torque driver.

FIG. 41 is a top isometric view showing adhesive being dispensed in recesses of the prosthesis.

FIG. 42 is a bottom isometric view of the abutments with attached copings in the process of being positioned into the recesses of the prothesis after adhesive is dispensed.

FIG. 43 is a top isometric view of the copings attached to the prosthesis after adhesive curing and pick-up.

FIG. 44 is a bottom isometric view showing the removal of temporary screw posts from the implant abutments after the pick-up process.

FIG. 45 is a top view of a drill in position to create a pilot hole in the prosthesis of FIG. 43.

FIG. 46 is bottom view showing the pilot holes from the process of FIG. 45 extending through the prosthesis to exit on the occlusion side.

FIG. 47 is a bottom isometric view showing a counterbore positioned on the occlusion side of the prosthesis to provide clearance for the definitive screw.

FIG. 48 is a bottom isometric view illustrating a reamer positioned to clean residue from the bore.

FIG. 49 is a bottom isometric view of the prepared prosthesis with definitive mounting screws prior to fastening to the abutments.

FIG. 50 is a top isometric view corresponding to FIG. 49.

FIGS. 51-52 are top and bottom isometric views schematically illustrating the material removed from an existing denture necessary with inventive concepts disclosed.

FIGS. 53-54 are top and bottom isometric views schematically illustrating the material removed from an existing denture necessary using prior art impression screw systems.

FIG. 55 is a top isometric view of a bite fork for holding a prosthesis.

FIG. 56 is a top isometric view of a prosthesis attached to a bite fork with bonding agent.

FIG. 57 is a schematic description of a process for preparing and placing a prosthesis for coping pick-up including robotic assistance.

FIG. 58 is an isometric view of a representative robot for dental implant surgery and prosthesis positioning for coping pick-up.

FIG. 59 is an isometric view of a robotic reference arm with splint.

FIG. 60 is an exploded isometric view of a robotic dental handpiece and bite fork.

FIG. 61 are top and bottom exploded isometric views of a bite fork and interface wafer.

FIGS. 63-64 are top and bottom assembled isometric views of the robotic prosthesis delivery jig of FIGS. 60-62 with an attached prosthesis.

FIG. 65 is a top isometric view of an open intaglio surface prosthesis with integral robotic dental handpiece interface.

FIG. 66 is a top isometric view of an open intaglio surface prosthesis with integral robotic arm interface.

### DETAILED DESCRIPTION OF THE INVENTION

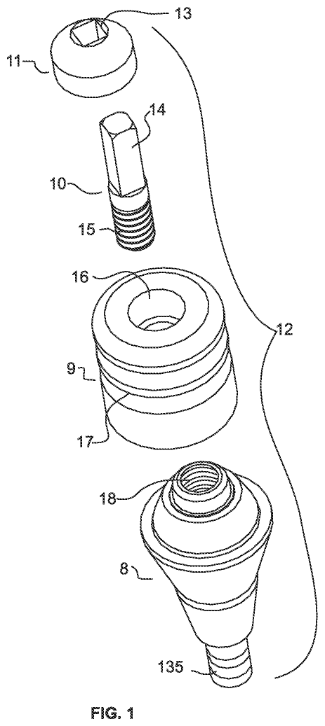

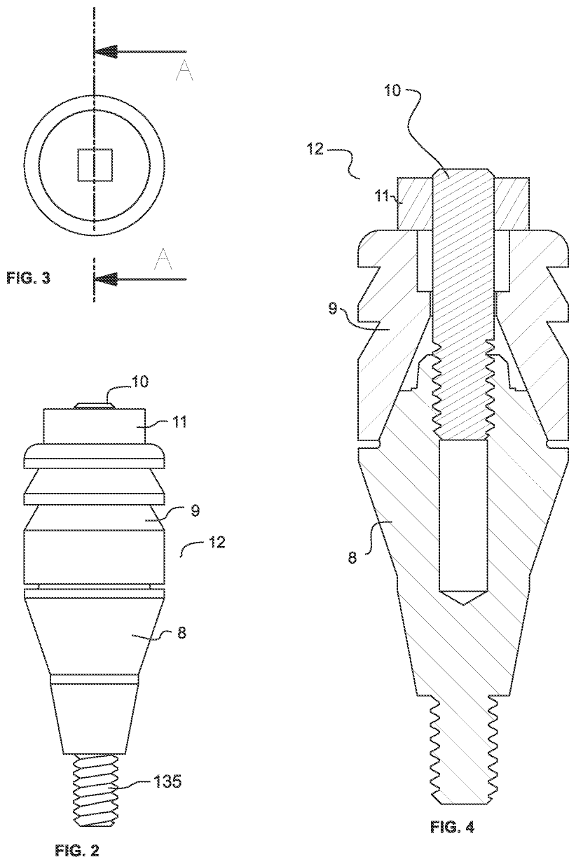

FIG. 1 shows an exploded view of one embodiment of a temporary alignment system 12 for use in a direct coping pick-up process for screw attachment of a prosthesis. Implant 8 has a distal end which may include attachment feature portion 135 illustrated as a screw thread for direct attachment to the patient's jaw and a proximal end with an abutment shaped to accommodate a coping 9. The abutment includes threaded section 18. The coping has a central bore 16 to accommodate screw fastening and may include ridges 17 or other structures or surface treatments to increase retention to the prosthesis with adhesive. The coping and abutment mating surfaces as shown in FIG. 1 are symmetrical. The inventive concepts of this disclosure may also be applied to abutment and coping systems that are keyed to restrict mating orientation. The coping may be mounted onto the abutment through rotation of a temporary adjustment screw comprising a threaded post 10 and cap 11. The cap 11 is mechanically attached to post 10 in a manner that allows relative axial motion with the application of a predetermined force. As illustrated in FIG. 1, cap 11 has a square central bore 13 that is press-fit onto a square portion 14 of post 10. The cap 11 temporarily retains coping 9 onto abutment 8 during the pick-up process of the coping into the prosthesis with a force aligned directly along the same axis as the semi-permanent screw that will be used for definitive prosthesis mounting. The portion of the post engaging the abutment threads also prevents any pick-up material from contacting the abutment screw threads. After the coping is picked-up into the prosthesis, the cap and coping may be released from the abutment by applying an axial force. In the embodiment shown, the post threads 15 remain engaged with abutment threads 18 after the pick-up process. The post can then be removed from the abutment by unscrewing it. The prosthesis with embedded coping can be subsequently processed to accommodate semi-permanent screw attachment as will be described later.

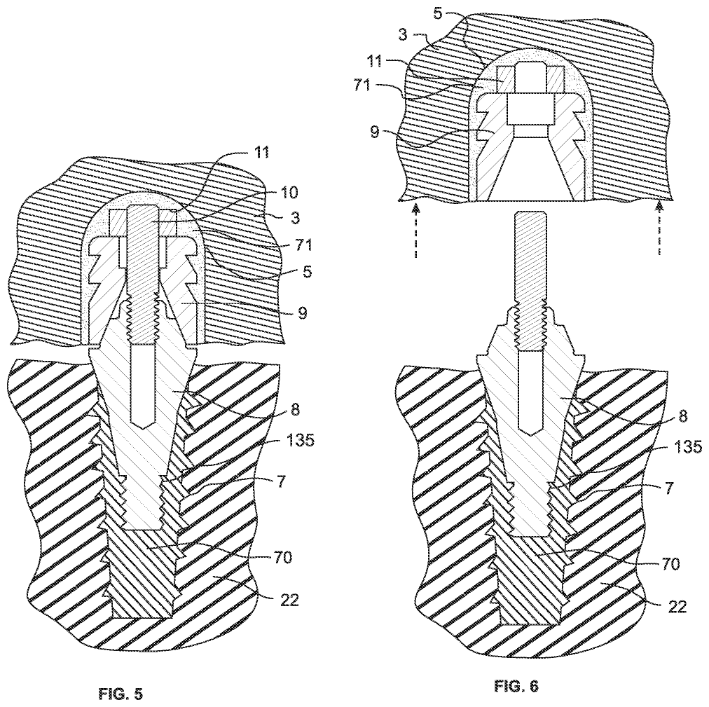

FIGS. 2 and 3 show a side and top view of the assembled system of FIG. 1; FIG. 4 shows a cross-sectional view along the axis as indicated. As shown in FIG. 4, matching conical surfaces on the exterior of the abutment 8 and the interior of the coping 9 are engaged. The coping is pulled into alignment by an axial force from the lower surface of the cap 11 pushing against the upper surface of the coping from screwing the threads of post 10 into the implant abutment. The distal end of the abutment may be directly attached to the patient's jaw or attached to a separate implant attached to the jaw. The inventive concepts are not dependent upon the nature of the implant, so the distal end attachment feature 135 is represented schematically.

The capability for relative axial movement between the cap 11 and temporary attachment post 10 allows separation of the temporary attachment post from the cap without tools after retaining the coping onto the abutment during fitting and bonding of the coping into the prosthesis. These parts are accurately aligned during the bonding process since the temporary post engages the abutment threads to provide an axial force holding the coping to the abutment just like the screw used for final attachment.

The cap may be sized to have an amount of press-fit mechanical interference to the temporary attachment post to provide an axial retention force of approximately 20 to 900 grams, this force being sufficient to retain coping 9 during assembly while allowing relatively easy pick-up removal of the coping and prosthesis from the temporary attachment post after assembly. The temporary attachment post 10 may include indicators (not shown) in the end by the cap 11 to provide intermediate visual and/or tactile feedback on the depth of threaded engagement of the post during screw attachment of the coping to the abutment.

In this embodiment, screw driving torque is provided by the square cross-section of the cap aperture and temporary attachment post. In the cross-sectional view of FIG. 4, the post 10 has been screwed in so that only a small, beveled surface of the post extends above the top of the cap when the post threads bottom out in the abutment. During installation, this optional configuration provides feedback to the dental practitioner when the torque increases as the threads bottom. The square drive tool engagement can also be designed to disengage at a pre-designed minimum extension of the square post above the top of the cap. Alternatively, a tool with a specified maximum torque may be used to ensure engagement of the coping and abutment surfaces.

The screw driving torque function may be accomplished by engaging other features on the temporary attachment post such as hex or spline or asymmetric features. Alternately, the cap may be used to engage a screwdriver or other tool for torquing. For example, in the case of a cylindrical temporary attachment post top portion, driving torque can be provided by the friction of the press-fit between the cap and temporary attachment post. For example, a medium press-fit of a 3 mm diameter 1 mm thick nylon cap onto a 1.3 mm diameter stainless steel cylindrical rod may produce approximately 500 grams of axial retention force and 17 gram-cm driving torque. A typical screw thread size for appliances is m1.4×0.3. The retention/axial force of the cap to the temporary attachment post may be determined by the degree of press-fit and frictional properties between the cap and temporary attachment post, and/or spring features incorporated into the temporary attachment post and/or cap.

In actual trial installations, polymer caps of approximately 1.2 mm thickness press fit onto posts of approximately 1.4 diameter with cylindrical axial bores have been sufficient for installation and pick-up removal of a single prosthesis from multiple implant abutments in which the axes of the screw threads in the implant abutments are not mutually parallel.

The separable mechanical attachment means may include any combination of techniques, including frictional forces from interference, adhesives, waxes, chemical bonding, solders, elastically or inelastically deformable spring, snap or interlocking structures, thermally or electromagnetically fusible materials, fracturing structures, etc.

FIG. 5 through FIG. 9 show example cross-sectional views of some of the different stages of installation of the temporary fastener system into a denture. FIG. 5 shows a cross-sectional view of the assembly of the first embodiment during the initial phase of the coping pick-up process. As illustrated, the implant abutment 8 is attached to a schematic implant 70. The implant 70 has an interface 7 (shown schematically) that is attached to the patient's jawbone 22. The coping 9 is held against the implant abutment 8 through the temporary screw comprising the post 10 and cap 11. A blind clearance hole 5 in the prosthesis 3 is sized to accommodate the coping and temporary screw. Pick-up material 71 is positioned in the clearance hole 5 to capture the coping in proper alignment within the prosthesis. After the pick-up material has set up, the prosthesis 3, coping 9 and temporary screw cap 10 assembly is pulled off the patient's jaw as shown by the arrows in FIG. 6. The coping 9 and cap 11 are released from the abutment 8 and post 10, while the threads of the post 10 keep it engaged in the implant abutment 8. The post 10 is subsequently removed to make the abutment threads 18 available for holding the prosthesis in proper alignment with a definitive screw.

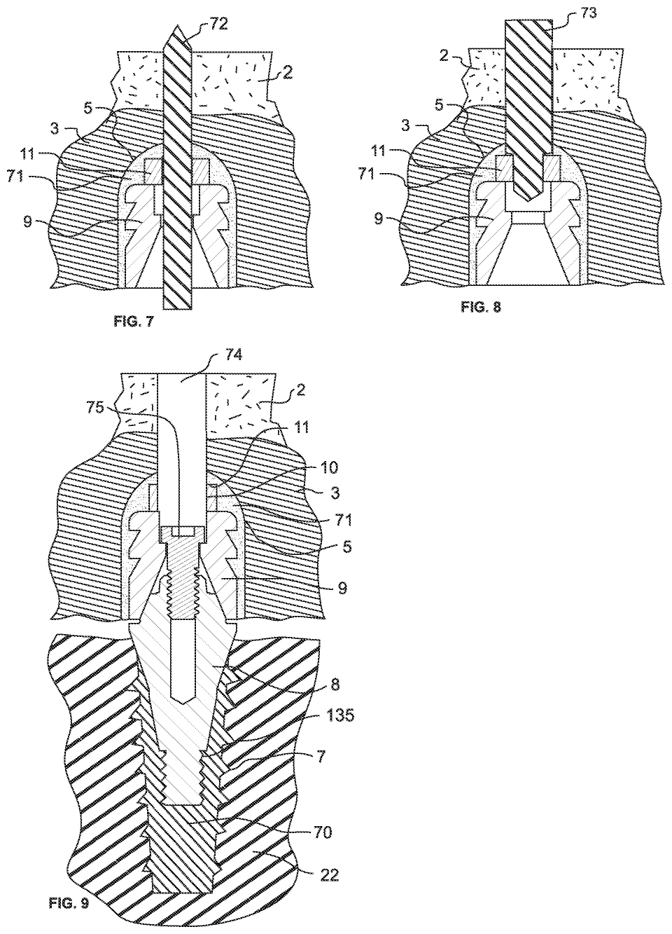

The prosthesis/coping assembly must be processed after the pick-up process for screw attachment. A pilot hole is drilled with bit 72 from the bottom side of the assembly. As shown in FIG. 7, the diameter of the bit may be selected to use the interior bores of the coping and cap to act as guides for hand processing. Alternately, a drill guide may be employed that engages a portion of the interior of the coping may be used to align the pilot hole. The pilot hole provides guidance for a tool to provide clearance for the definitive screw shaft and head. FIG. 8 shows a stepped drill that cuts both clearances in a single process. Alternatively, the shaft and head clearance may be drilled with two drills in two process steps. If desired, the cap 11 of the temporary screw can be fabricated of a material that is easier to drill than the coping material to provide feedback on the drill position. It has been found that it is possible to distinguish where the drill is positioned by feel at the interfaces between the adhesive and the top of the cap (as shown in FIG. 8) and the bottom of the cap and the top of the coping. Since pick-up material was blocked from entering the coping, drilling resistance decreases rapidly upon breaking through the bottom of the cap and entering the interior channel of the coping. Color differences in the materials can also be detected. Alternatively, a tool can be configured to fit within the bore of the coping to limit the depth of the drill.

Once clearance for the definitive screw has been made from the top of the prosthesis, the prosthesis is ready for mounting to the implant with definitive screw 75 as shown in FIG. 9. The screw clearance hole in the prosthesis may be filled with Teflon tape and color-matching composite materials for aesthetic purposes. In the illustrated embodiment, the coping bore has a shoulder to engage a flat surface on the underside of head of the screw. Alternate configurations are possible without deviating from the temporary screw system for coping pick-up disclosed.

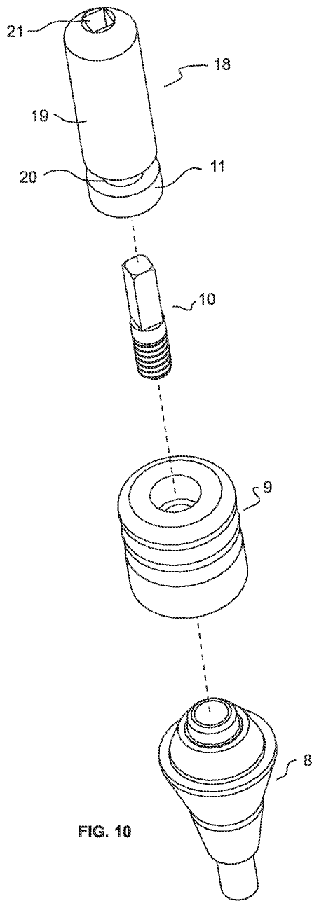

FIG. 10 is another exploded isometric view of parts of an embodiment of the invention. In this example, cap 11 is integrated into a breakaway installation tool 18. The tool portion 19 may be used to install the temporary attachment post 10, and then section 19 broken away at mechanically weak separation feature 20, for example, when the post bottoms out in the abutment leaving the cap portion 11 in position. Alternatively, the tool can be designed to increase stress on the separation feature as the top of the post moves axially down relative to the weakened section. As illustrated, after the cap portion 11 breaks away from the tool portion 19, drive feature 21 in the tool portion may be used to remove the post 10 after the pick-up process. One or both ends of the tool may be shaped to subsequently engage and remove the temporary attachment post 10 after installation of coping 9 into the denture.

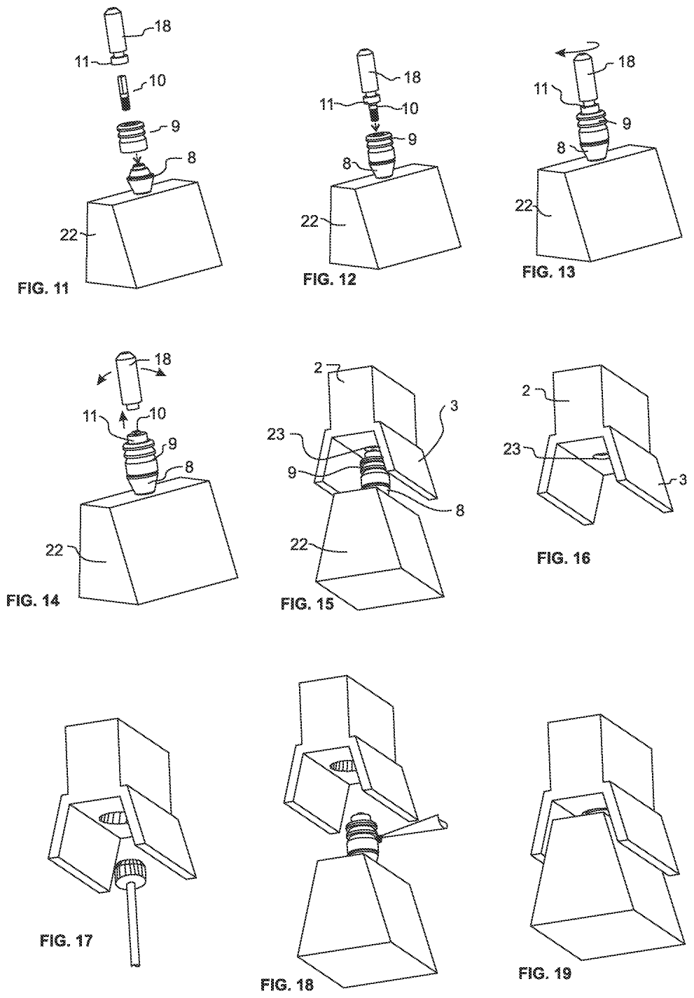

FIGS. 11-26 provide in schematic isometric views the process steps for using the elements of an exemplary embodiment introduced in FIG. 10 for a single implant system such as a crown. In FIG. 11, a schematic isometric view of a portion of the patient's jaw 22 is shown with an implant 70 (not shown) installed, and abutment 8 installed into the implant. The coping 9 is placed onto the abutment 8, the top portion of temporary attachment post 10 is assembled onto cap 11. The top portion of temporary attachment post 10 and cap 11 are configured such that there is a means to drive the temporary attachment post into the abutment, while allowing axial movement of the cap 11 relative to the temporary attachment post axis, the cap/temporary attachment post interface having sufficient retention force to keep the coping in place on the abutment during subsequent installation steps, and the temporary attachment post being removable after picking up the coping into the prosthesis. Cap 11 may be attached to a break-away installation/removal tool 18.

FIG. 12 shows the coping 9 placed on the abutment 8 and the cap/tool 18 installed onto temporary attachment post 10. The coping-abutment surfaces may comprise a conical or spherical concave feature on the coping 9 that mates with a complementary feature on the top of the abutment 8, as illustrated. Alternatively, keying features may be employed to restrict relative rotational orientation, particularly in the case of single implants.

FIG. 13 shows the temporary attachment post being threaded into the abutment 8, through a clearance hole in the coping 9. The cap abuts the coping to secure the coping to the abutment. The cap 11 is free to move axially along the temporary attachment post with a frictional retaining force of the cap to the temporary attachment post. A known amount of retention force of the coping to abutment is provided by the design and materials used in the cap and temporary attachment post.

FIG. 14 shows the tool portion of the cap being removed after the cap has broken away; the tool may be used for removal of the temporary attachment post at a later step.

FIG. 15 shows marking of the position of the coping onto the appliance for drilling a recess for the coping in the prosthesis.

FIG. 16 shows the prosthesis with coping drilling location marking 23.

FIG. 17 shows the prosthesis after drilling the cavity for the coping; the cavity for the coping may be accurately drilled slightly larger than the coping, without significant unwanted material removal from the prosthesis.

FIG. 18 shows pick-up material being applied to the coping and/or prosthesis after confirmation of freedom to provide proper occlusion during dry testing. Although not illustrated, the cavity of the prosthesis or the coping may optionally include features to provide venting of excess pick-up material if desired.

FIG. 19 shows the bonded coping being picked up into the prosthesis.

FIG. 20 shows the prosthesis removed from the temporary attachment post; the temporary attachment post/cap design allows removal of the prosthesis from the temporary attachment post. The coping is now incorporated into the prosthesis.

FIG. 21 shows the temporary attachment post being removed with the tool portion of the cap.

FIG. 22 shows the abutment installed in the implant with the temporary attachment post removed.

FIG. 23 shows drilling a small guide-hole (for example 1-2 mm diameter), through the clearance hole of the coping, into and through the prosthesis. This guide hole provides a small reference hole for enlargement of the hole to accommodate the prosthesis retaining screw.

FIG. 24 shows enlarging the guide hole to a clearance hole for prosthesis retaining screw, approximately the diameter of the head of the retaining screw (e.g. 1.5-2.5 mm). The clearance hole would typically be drilled down to the top surface of the coping.

FIG. 25 shows the prosthesis being installed onto the implant abutment by placing the prosthesis coping onto the abutment, and installing the prosthesis retaining screw.

FIG. 26 shows the assembled prosthesis on the implant.

FIG. 27 contains a summary of a generalized process extending the basic process described above for a prosthesis attached to multiple implant abutments.

Another embodiment of a prosthesis and implant alignment system and tool are shown in FIG. 28. In this case, the cap is in the form of a hex nut 63 that is press fit onto alignment post 62 to act as the temporary alignment screw. The drive tool 64 includes a concave hex socket 68 that fits temporary post nut 63, and coping retaining portion 69 that retains coping 61 with a slight interference fit. Thus, as shown in FIG. 29, the temporary screw comprising nut 63 with post 62 is engaged with the socket portion of the tool, and the coping 61 is retained in the tool. The coping is seated onto the abutment 60 by using the hex portion of the driver to rotate the nut and engage the threads 66 on the temporary screw post 62. FIGS. 30 and 31 show an exterior and cross-sectional view of the mounting of the coping on the abutment with the tool. The tool may also have an integrated feature for removing the post as shown in FIG. 32, such as the opposite taper-fit end feature 70 shown in cross-sectional view FIG. 33. FIG. 28 illustrates optional undercut 67 for stronger adhesive locking. Optional anti-rotation flat 65 is shown Such an anti-rotation flat may be used on mating surfaces in circumstances in which there is a preferred orientation around the axis of the screw threads.

Another embodiment of the inventive concepts in which the cap of the temporary screw is shaped to engage the driving tool is shown in FIGS. 34-36. Cap 102 is mechanically attached to alignment post 101 at end portion 101A to form temporary alignment screw 100. This mechanical attachment may result from press-fitting a polymeric cap 102 onto a metal post 101 to provide adequate resistance to rotary slip to temporarily attach the abutment to the implant while still allowing axial movement during the pick-up process. A hexalobular internal (Torx) drive feature is shown in the end of the cap 102, although other bit socket shapes are possible. As previously discussed, other mechanical engagement means besides an interference fit are possible. The post may be made of different materials than these, or even be of unitary construction with weakened sections that fracture under a desired pick-up axial force.

In a preferred embodiment the post 101 is made from stainless-steel or titanium, and the cap of polymer such as PEEK or acetal. The short length of the post and threaded fastener in this embodiment allows separation at high degrees of angularity of the assembled parts in use. This tolerance for off-axial removal has been found to be particularly advantageous when the prosthesis is to be definitively screw mounted to multiple implant abutments.

The general process for converting an existing removable denture for definitive screw attachment onto four implants with this embodiment is illustrated in FIGS. 37-50. This prosthesis may be, for example, a removable denture that was used prior to implant surgery or a duplicate of such an existing denture as described in U.S. Provisional Patent Application 62/774,402 incorporated herein in its entirety.

FIG. 37 shows a schematic representation of a human jaw portion 106 with implant abutments 107 installed. It does not matter for this discussion if the abutments are separable from the implanted portion of the implant or not. Prosthesis 103 is shown with occlusion side 104 and intaglio side 105. Pick-up marking caps 108 are installed onto abutments 107. The location of abutments 105 is marked onto the prosthesis using customary methods by mating the prosthesis with the abutments. FIG. 38 shows the abutment positions 109 marked onto prosthesis 104. FIG. 39 shows drilling of blind holes 110 slightly larger than copings 112 in marked locations, with burr tool 111.

FIG. 40 shows the installation of copings 112 onto abutments 107 using separable fastener assembly 100, and torque driver 113. The torque driver 113 prevents over-tightening of temporary screw 100 and possible separation of the cap 102 from the post 101 due to rotary motion. The cap 102 and/or post 101A of the separable fastener 100 may be mechanically captured or adhered to the coping 112, or may be designed to loosely fit into the bore of the coping as illustrated with axial force from tightening the separable fastener holding the coping to the abutment 107. The prosthesis is placed over the mounted copings to ensure proper fit.

FIG. 41 shows application of acrylic or other adhesive into cavities 110 of prosthesis 103, which is subsequently fitted onto copings 112.

FIG. 42 shows prosthesis 103 being mated with copings 112. After the adhesive sets, the separable fasteners allow easy removal of the prosthesis from the abutments with the copings incorporated into the prosthesis. The angular tolerance for removing the prosthesis from multiple abutments allows applying pick-up forces sequentially around the edge of the prosthesis to work the caps 102 off the posts 101. The caps 102 remain in the prosthesis with the copings 112, while the posts 101 remain in the abutments.

FIG. 43 shows the prosthesis with incorporated copings 112 after the pick-up process.

FIG. 44 shows the removal of the threaded post 101 from the implant abutment with removal tool 115. This allows the implant abutment threads to be accessible for subsequent definitive screw attachment.

FIG. 45 shows the drilling of the small pilot hole 117 (e.g. using a drill bit 116 of approximately 1.4 mm diameter), from the intaglio side 105 of the prosthesis out through the occlusion side 104. It has been found that the bore of the coping 112 and the bore of the cap provide adequate guidance for this hole, although a tooling guide could readily be designed to mate with the particular coping used. The pilot hole is drilled completely through the prosthesis to the occlusion side 104 as shown in FIG. 46.

FIG. 47 shows enlarging of the pilot hole 117 to allow clearance for a prosthetic mounting screw 121. The clearance holes 119 are drilled down to the top of the coping using counterbore drill 118. This requires only a small diameter enlargement (for example, approximately 2.4 mm).

FIG. 48 shows a step of a final hand reaming with reamer 120 of the coping bore to clean out any debris or remaining material from the cap that would interfere with the definitive screw.

FIGS. 49 and 50 show the final installation of prosthesis 103 onto abutments 107 using definitive prosthetic screws 121. The screw holes may be subsequently filled to use the modified prosthesis depending upon the anticipated use as a short-term or more permanent prosthesis. This sequence of process steps essentially follows the material provided in FIG. 27.

Note that in the above procedure very little material is removed from the prosthesis during the coping pick-up installation process. The boring process in FIG. 39 need only be sufficient to provide clearance for the coping and temporary screw. Angular variation in the axes of the implant abutments does not appreciably increase the size of the cavity boring required in this closed tray process compared to the additional prosthesis material that must be removed with relatively long impression screws and sleeves in a conventional open tray conversion process for definitive screw attachment. FIGS. 51-52 provide a schematic comparison of the size of the recess borings required using the concepts disclosed compared to the prior art conversion process with larger through holes in FIGS. 53-54. In addition to the stubby embodiment shown in FIGS. 34-36, the parent U.S. patent application Ser. No. 15/596,361 incorporated by reference in its entirety includes other separable fastener embodiments and associated tools that may be beneficially used with the processes described in the present application. These are not the only examples of means for provisional coping attachment. The inventive concepts disclosed are not meant to be restricted to a temporary attachment post with standard screw threads that both engage and disengage the threads in the implant abutment through rotations. For example, alternate separable temporary attachment screw posts embodiments are possible providing features that allow the post to removably hold the coping to the abutment by other means than a separable cap. Other means for provisional coping attachment are also known in the industry including various forms of snap-on devices that are not used with definitive screw attachment. Embodiments presented that do not specifically rely upon means for provisional attachment to an implant abutment using screw attachment should also be considered to be within the scope of the inventive concepts.

A desirable feature of disclosed embodiments is the relatively small size possible for the separable fastener itself as well as the coping and implant abutment. This relatively small size reduces the amount of material that must be removed from the converted prosthesis for clearance not only from angular variation in the axis, but also in the width and length of the components. In the case of preparing a new denture for coping pick-up and screw attachment, smaller dimensions may provide more efficient cycle times for milling the smaller clearance bore in a new custom or stock denture for the pick-up.

Reduced material removal to convert the existing prosthesis to accommodate the smaller separable fasteners retains more of the structural integrity of the prosthesis through the milling and fitting processes. Avoiding distortions in the prosthesis from handling helps maintain proper occlusion and soft-tissue contact of the original system. Since the disclosed separable fasteners allow the converted denture to be used in a closed-tray pick-up process, additional structural support during milling or the pick-up process can be provided with the arch bar or bite fork 150 shown in FIG. 55. The bite fork 150 illustrated is an arched plate constructed of sheet metal or other stiff material including titanium, cobalt chromium, stainless steel, PEEK, PEK, PMMA or other metals, alloys or resins with known processing techniques such as additive or subtractive machining, molding, casting, etc., sized to support a prosthesis. A user grip 153 is provided on the end for the dentist. As shown in FIG. 56, the prosthesis 103 can be temporarily affixed to bite fork 150 with an adhesive 152. The adhesive 152 provides a temporary bonding between the prosthesis 103 and the bite fork 150. Any of the temporary fixing or impression materials known in orthodontics may be used to temporarily hold these elements together for processing. The assembly formed by bonding these elements together stabilizes the denture and prevents it from distorting during the milling process that creates clearance for the implant abutments 107, copings 112 and temporary fasteners 100. After milling, the optimum sequence of separation of the components during the overall conversion and pick-up process may be chosen depending upon particular circumstances of the case. That is, the prosthesis and bite fork assembly can be removed from the patient's mouth together, or the bite fork can be removed before the prosthesis is removed through the release of the separable fastener. Obviously, the prosthesis would need to be removed from the bite fork and adhesive to check proper occlusion with opposing natural or prosthetic teeth.

A bite fork with attached prosthesis may also be advantageously employed as a delivery jig in robotic assisted dental implant surgery and immediate new prosthesis fitting. The processes for fitting a prosthesis for multi-abutment screw attachment described above and summarized in FIG. 27 may include a dependency on the alignment skills of the dental practitioner. The fitting of the prosthesis to the implant abutments for the pick-up process requires that clearances are sufficiently large to provide passivity, but not so large that the prosthesis is not in the ideal position for the patient. This tradeoff in proper clearance may cause multiple trial fittings until enough prosthesis material is removed. A method to reduce the human element to reduce chair time for the patient and improve alignment outcomes with a modified pick-up process with the separable fasteners is provided in FIG. 57.

The embodiment described is an extension of a digital workflow planning and processing using robotic assistance systems for implant placement and prosthesis design such as the Yomi System from Neocis. FIG. 58 shows a 6-axis robotic system 220 used in robotic-assisted dental surgery, such as in the for drilling and placement of dental implants. A dental handpiece base 203 is attached to robotic actuator arm 201 with handpiece arm interface 202. As shown in FIG. 60, handpiece base 203 generally has a detachable/interchangeable head assembly 204 to allow varied torques, speeds and tool attachment. Cutting tools and bits are installed into the end 205 of head-assembly 204. A common interface standard for dental handpiece tools such as burrs or drills, etc. is a 3/32″ diameter latch-lock shank. The form of attachment between these tools and delivery jigs described below is a design choice, but generally, the translational and rotational spatial relationship of tools relative to the robot must be known by design or measurement after attachment. Attachment of devices to the robot may be done with mechanical, magnetic, pneumatic or other techniques known in the machine tool industry.

For robot/patient planning and positioning, some current procedures utilize a splint 301 shown in FIG. 59 rigidly fixed to the patient's jaw 303 with a removable radiopaque fiducial plate (not shown) attached to the splint to reference and track placement of surgical procedures and planning and compensate for movement of the patient during the procedure. The splint 301 is attached to the patient's jaw and to a reference arm 221 in communication with the robotic system at separable interface 222. Cone-beam computed tomography (CBCT) scans are made with the splint 301 and fiducial array in place, thus providing a reference for digital design and location-tracking. A feedback positioning linkage with fixed mounting replaces the fiducial plate and attaches to the splint 301 and robot system 220 through the reference arm 221 to provide location and tracking of the patient's movement and orientation and guiding of tools attached to the actuator arm 201. For example, splint position encoders 302 shown schematically in FIG. 59 may provide information to the robotic system on the current position and orientation of the splint 301 which provides direct information on the position of the patient's jaw 303. In this way, the robot is spatially aware of the position of the jaw in real time to properly apply tools affixed to the dental handpiece 203. The inventive concepts may also be adapted to other systems that use alternate techniques to fix or dynamically monitor patient position relative to the actuator arm such as some form of dynamic imaging. For convenience, the description describes a process for placing a mandibular prosthesis, but can be equally applied to placing a maxillary prosthesis. While robotic assistance with haptic or other sensory feedback is described, autonomous processing of one or more steps by the robotic system is an obvious variation considered to be part of the inventive concepts disclosed.

FIG. 60 shows how the dental handpiece base 203 may be adapted to position a prosthesis delivery jig for pick-up processing using the spatial awareness of the robotic system. The prosthesis delivery jig illustrated in FIG. 60 comprises a bite fork 155 with an adapter block 305 for attachment to the handpiece end 205. Features in the adapter block 305 for repeatable alignment can include alignment hole 306 for interface pin 304 and attachment screws 307.

For some processes, the robotic assistance and spatial awareness may depend upon the operator first providing input to the robot. For example, the operator may guide the actuator arm of a robot through a path in virtual or real space for presenting a prosthesis delivery jig into rough position in a dry fit process. In the subsequent actual pick-up process, the robot could provide assistance by constraining movement to an envelope around this path relative to the actual patient position. Another form of robotic assistance from motion constraint that may be employed is preventing the operator from moving the prosthesis after presentation to the coping being picked-up until a prescribed time has elapsed necessary for bonding agents to set.

After the patient has been scanned with the splint and fiducial array and the relation of the fiduciary array with the software completed, a digitized prosthesis is imported and positioned in the ideal position as prescribed by the restorative team. The digitized prosthesis can be from a digital library of off-the-shelf dentures 206 as shown in FIGS. 63 and 64. The schematically illustrated denture 206 is a representative example of an open intaglio surface prosthesis which has been selected to require minimal or no material removal due to interference with the implants and copings for pick-up. Only a limited number of mass-produced impression tray sizes and shapes are needed to fit a large population of patients. In a similar manner, the disclosed process provides increased standardization of mass-produced prostheses for rapid processing for screw attachment after coping pick-up processing.

While no clearance boring is necessary in the extreme case of a prosthesis with an intaglio surface resembling an impression tray, there are still advantages in adapting this process when more solid standardized or custom prostheses are used. In these cases, the position and size of the clearance bores does not need to be as accurate since the robot is placing the prosthesis. The clearance customization of the prosthesis can be prepped in advance of implant surgery based upon the robotic workflow planning software. Inaccuracies introduced in the actual implant positions relative to the digital plan can be considered in oversizing the bores in preparation of the prosthesis. Final prosthesis position in the patient's mouth will depend upon the inherent accuracy of the robotic positioning, not the nesting of elements held in place by human judgement. Mechanical uncertainties in a loose nesting of bore holes and inaccuracies in actual versus planned abutment placement may be compensated by the displacement and flow of the bonding material before it sets.

The process of FIG. 57 exploits the known position of the robot actuator arm relative to the splint-attached robot reference arm that remains attached to the patient that was used for the implant surgery. Mass-produced dentures may be mounted in a known position by indexing features of the delivery jig, or a digitized custom prosthesis may be mounted and then scanned by intra- or extra-oral scanner for determining position on the delivery jig. The requirement is of course that the prosthesis is mounted onto the robot arm, by some means, in a known position and orientation relative to the robot's known position with final accuracy sufficient for the pick-up procedure (approximately within a tolerance zone of 0.5 mm). A prosthesis 206 is mounted to the bite fork 155 in a pre-determined position or may be imaged with fiducial markers fixed in the prosthesis or bite fork assembly to determine position. By mounting the bite fork with prosthesis on the robot actuator arm in a known position, the robotic system can determine where the prosthesis is located relative to the robotic actuator position in the same way that the system knows how a drill or implant is positioned relative to the robotic actuator arm.

Note that the robot reference arm 221 does not need to be removed from the splint 301 unlike a process using the robot to bore clearance holes of sufficient size in a known position in a prosthesis to accommodate the implant abutments, copings and temporary fasteners for the pick-up process. This boring process necessarily requires that the reference arm be disconnected from the patient and attached to the bite fork in order to process the prosthesis through a material removal process. The prior art describes how these clearance borings could be used to manually fit the prosthesis in place. The clearance holes must be sized to allow for uncertainties in actual implant position versus planned implant position and actual bore size versus planned bore size. More accurate positioning may be accomplished by also using the robotic system to position the prosthesis by using the robotic actuator arm to position the prosthesis in the patients mouth and using the pick-up bonding agent to accommodate implant or boring tolerances. While this robotic assisted positioning could be done with a prosthesis with material removed for abutment, coping and separable fastener clearance, the process in FIG. 57 in which material is added to an open intaglio surface prosthesis though the pick-up process provides additional benefits in cycle times and standardization.

Through the patient position information through the reference arm splint, the position of the prosthesis relative to the patient can be determined. As a result of this positional information, the robotic system can be used to place the prosthesis on the copings for pick-up without any dependence on human judgement and without a dependency upon the close nesting of pick-up copings in complimentary bore holes in the prosthesis. Even in a prosthesis conversion or fitting process in which material removal is necessary to avoid mechanical interferences, less precision is required since final prosthesis positioning in the patient's mouth is not dependent upon accurate milling of clearance bores for tight nesting with the abutment assemblies for coping pick-up.

FIGS. 60-64 illustrate an embodiment of a prosthesis delivery jig for robotic positioning of a prosthesis 206 for pick-up processing. In an illustrative embodiment, the delivery jig comprises a bite fork 155 and robot attachment adapter block 305. In this example, adapter block 305 mechanically attaches to the handpiece end 204 that is mounted to the robot actuator arm 201. FIG. 60 shows the adapter block 305 assembly and bite fork 155 prior to being attached to the handpiece end 204. The handpiece in this example is attached to the robotic arm in a known position and orientation for use by the robotic system software and hardware. In this example, adapter block 305 is located and retained onto handpiece end 204 using a contoured surface that fits the body of the handpiece end 204, and a latch-lock pin 304 that installs and locks into the normal tool holder/collet 205 of the handpiece and fits into alignment hole 306. Attachment screws 307 may be used to further lock the adapter block 305 to handpiece 204. The bite fork 155 is attached to the robot in a known position through the adapter block 305. A pattern of locating holes 154 may be included on the bite-fork for registration of the prosthesis onto the bite-fork or to provide imaging reference points. The bite fork and adapter block assembly may made as one piece from polymers and or metallic materials. Alternately the bite fork 155 may be removably attached to adapter block 305.

Through design data or through imaging, the position of the bite fork 155 and adapter block 305 assembly are known relative to the existing position and coordinate system of the robot and handpiece, allowing for guiding and placement of the prosthesis. To facilitate the use of off-the-shelf prefabricated arches, interface wafers 308 shown in FIGS. 61-64 may be utilized. The interface wafers 308 provide a similar functionality to the prosthesis adhesive 152 shown in FIG. 56 in fixing a prosthesis to a bite fork delivery jig. The wafers 308 include locating features 309 that mate with reference features on the bite-fork. These locating features may be used with adhesive attachment or may incorporate snap fit features. On the opposite side of the wafers 308, tooth interface features 310 may be used to locate and retain the arch during the pick-up procedure. Thus, by installing an interface wafer 308, the position of the arch relative to the bite fork is known, and the interface wafer retains the denture to the bite fork during the pick-up procedure. In this manner, the same bite fork 155 and adapter block 305 assembly can be used with a universe of different prostheses by using the appropriate interface wafer for each prosthesis.

FIGS. 63-64 show a prefabricated arch 206, installed using interface wafer 308 onto the bite fork 155 and adapter block 305, and the adapter block installed onto handpiece end 204. Discrete interface wafers may be fabricated to align all the available prefabricated arches. The interface wafers 308 may be injection-molded, cast, printed etc. from polymers or elastomeric materials. For custom dentures or other prosthetics, a compliant curable registration material may be applied to the bite fork and the prosthetic occlusal surfaces indexed to the fork while the registration material sets similar to the description of FIGS. 55-56. In order to reference the custom denture to the bite fork, the locating features 154 or other fiducial features may be imaged along with the denture. This scanned information can be imported and processed through methods known in the robotics dental industry to both digitally simulate and physically manipulate the denture relative to the patient. A bite fork, or similar fixture plate, may be used with auxiliary mounting fixtures to help locate the arch in a known position with respect to the bite fork and with respect to the robot.

Alternatively, the custom prosthetic can be designed in a design software in relation to the bite fork and a custom interface wafer designed to include features that would index the custom prosthetic to the bite fork, for example, by including posts that fit into locating holes 154. The designed prosthesis, wafer and digital bite fork can be exported from design software and imported into implant planning software. The bite fork may be fixed or detachable but indexed to the attachment apparatus to maintain a consistent re-positioning of the bite fork. The bite fork may be made of a metal or high-performance polymer intended for re-use. The interface wafers may be intended for single use, but may also be made of a re-usable material (e.g. stainless steel, polyetheretherketone, etc.) The posts 309 may include snap features to lock onto the bite fork 155.

FIGS. 63-64 illustrate how the denture 206 is held on the bite fork 155 by the interface wafer 308 and the implant handpiece 203 used as the positioning reference to position the prosthesis in patient's mouth in the correct vertical and anteroposterior positioning, along with the correct pitch, roll and yaw. With the use of the haptic technology to position the prosthesis, the implant components can be picked up utilizing separable fastener technology while the robot arm maintains the proper position of the prosthesis during the curing of the pick-up material.

Once the pick-up material is cured, the sequence of releasing the prosthesis from the bite fork assembly and the prosthesis from the implant abutments is a design choice. In some cases, it may be desirable to allow the prosthesis to be retained on the abutments temporarily before completing the modification for screw attachment using the latter process steps from FIG. 27 at a future appointment. In this case, the prosthesis must release from the interface wafer with a lower force than would cause the separable fastener to separate. Although a single prosthesis attached to the delivery jig has been described, extending the process to robotically position mating prosthesis simultaneously is possible. For proper occlusion, however, the bite fork plate would need to be removed from between occluding teeth. The structural support for the jig would need to be located inside the arch and in the gum of the prosthesis for aesthetic reasons. The positioning relative to the patient of the assembly would be controlled by the side with the splint and robotic arm reference, while the patient would close on the other side. The two prostheses would be held in proper occlusion at least until the bonding agent sets.

While the discussion above has focused on how the robotic system and integrated workflow can be used to properly position and hold a prosthesis for a coping pick-up process more accurately than human judgement, the robot may also be used to help release the prosthesis from the implant abutments after the copings are bonded. The robotic system can guide the release of the separable fasteners by determining the appropriate force vector direction. The robot can also be programmed to restrict the range of motion so that upon release, the prosthesis and bite fork do not move more than necessary. In that sense, the robot can apply a controlled braking force when the separable fastener connection releases. In addition to these human-assisted benefits, the robotic actuator can be programmed to rock or vibrate in any direction to assist in the separation of the separable fasteners. The frequency and amplitude of these motions can directed more accurately and be much faster in frequency and smaller in amplitude than can be done by a human. The optimum movement will depend upon the geometry of the implant array and the mechanical and material properties of the separable fasteners and the fit of the copings and implant abutments.

FIG. 65 shows an example of an arch with an integral frame 219. Shown is a handpiece retention feature 311 with latching and orientation features similar to adapter block 305. In this embodiment the position of the prosthesis to the robotic arm interface is known from the manufacturing of the assembly. The frame may be integrally fabricated with the arch for example through a molding process. After pick-up processing, the frame may be cut away or include break-away or other features to support separation of the prosthesis portion 314 from the support portion 312. A variant of this embodiment is shown in FIG. 66 in which an integral prosthesis and delivery jig 315 includes a delivery jig arm interface portion 316 that is attached to the robot arm 201 at the same position that dental handpiece arm interface 202 is attached in FIG. 58 Other combinations of elements having known physical relationships may also be used with the robotics positioning for pick-up described above and are considered within the scope of this disclosure.

The embodiments of systems and methods to mechanically locate the arch by attachment to the existing handpiece for the pick-up procedure have been provided to illustrate inventive concepts. Other methods of locating arches relative to a robot arm and patient include optical scanning, contact probe measurement, real-time radiographic imaging (e.g. x-ray, NMR), electromagnetic field sensors, embedded integrated circuit position and movement sensors, passive sensors such as electromagnetic field sensors, embedded RFID type devices, etc. The delivery jig, bite fork, prosthesis, interface wafer and any other component used in the systems and methods may include geometric features, radio-opaque elements, or other elements to increase imaging contrast to assist determining spatial relationships through scanning.

Various embodiments have been described to illustrate the disclosed inventive concepts, not to limit the invention. Combining inventive elements of one or more of the embodiments with known materials, components and techniques in dental science to create further embodiments using the inventive concepts is considered to be part of this disclosure.

What is claimed is:

1. A robotic-assisted prosthesis delivery method for coping pick-up comprising:
affixing a coping to an implant abutment with a means for provisional fastening,
mounting a prosthesis onto a robot,
applying a bonding agent to at least one of the coping or the prosthesis,
positioning the prosthesis on the coping in a predetermined position,
maintaining the relative position of the prosthesis and coping with robotic assistance for a predetermined minimum time for bonding the coping to the prosthesis.

2. A robotic-assisted system for presenting a prosthesis to an implant abutment system comprising:
a robotic system,
a delivery jig attached to the robot,
a prosthesis, wherein the prosthesis is attached to the delivery jig in a known position, and
wherein the robotic system is spatially aware of the difference between the current position of the prosthesis to a predetermined mounting position of the prosthesis on the implant abutment system.
